(19) United States
(12) Patent Application Publication
(10) Pub. No.: US 2005/0154232 A1
(43) Pub. Date: Jul. 14, 2005

(54) Nitrosodiphenylamine derivatives and their pharmaceutical use against oxidative stress pathologies

(76) Inventors: Claude Lardy, Lyon (FR); Didier Festal, Ecully (FR); Lidia Caputo, Lyon (FR); Daniel Guerrier, Saint Genis Laval (FR)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 10/507,107

(22) PCT Filed: Feb. 12, 2003
(86) PCT No.: PCT/EP03/01370

(30) Foreign Application Priority Data
Mar. 11, 2002 (FR) 02/03025

(51) Int. Cl. C07C 243/06; C07D 207/46; C07D 211/26

(52) U.S. Cl. 564/112; 546/229; 548/571

(57) ABSTRACT

Compounds of formula (I) in which each of the phenyl rings represented is optionally substituted one or more times; n represents an integer selected from 0, 1, Z, 3, 4 and 5; W represents —CO— or —SO—₂; Z represents H; alkyl; aryl; or arylalkyl R₁ represents any monovalent organic group; and the pharmaceutically acceptable salts thereof, can be used in the treatment of pathologies that are characterized by an oxidative stress condition.

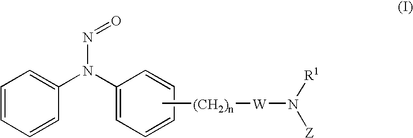

The invention relates to nitrosodiphenylamine derivatives, to pharmaceutical compositions comprising them, and to their use for preparing medicaments that can be used for treating pathologies that are characterised by an oxidative stress condition and a lack of availability of endothelial nitrogen monoxide (NO.).

Nitrogen monoxide (or nitric oxide NO.) is an important mediator in the physiology of cardiovascular, immune and central and peripheral nervous systems. It acts, among other mechanisms, by activation of guanylate cyclase.

Its action is ubiquitous: it is vasodilatory and gives a basal tonus to the entire vascular system. It has anti-clotting activity: its production by normal endothelial cells inhibits the formation of a thrombus. It is anti-proliferative, especially on the smooth muscle cells underlying the endothelial cells. It also inhibits the adhesion of monocytes to the vascular wall and, consequently, its conversion to a macrophage. It regulates endothelial permeability.

There is thus, in the physiological state, a situation of equilibrium between the production of free-radical species and the availability of NO.

Disequilibrium of this balance, the result of which is an excess of super-oxide anions in the face of a lack of NO, leads to the development of many pathologies.

Oxidative stress is caused by many factors, for instance hyperglycaemia, dyslipidaemias (production of oxidised, highly atherogenic “low-density” lipo-proteins (LDL)), hypoxia, insulin resistance, atherosclerosis, revascularisation techniques (including angioplasties with or without a stent), chronic rejection after transplantation, the majority of inflammatory processes, and smoking. Oxidative stress is characterised at the vascular level by an increase in free radicals, in particular of superoxide anions (O₂.⁻).

These O₂.⁻ radicals are capable of trapping the NO produced endogenously by the endothelial cells to form free-radical species that are even more deleterious, for instance peroxynitrites.

Among the pathologies concerned by a lack of production of endothelial nitrogen monoxide and/or an increase in tissue oxidative stress, mention may be made of (Recent Progress in Hormone Research (1988), 53, 43-60, table V):
atherosclerosis-associated ischaemias (lipid peroxidation, development, progress and rupture of atheroma plaques, platelet activation);
restenosis after angioplasty;
stenosis after vascular surgery;
diabetes;
insulin resistance;
retinal and renal microvascular complications of diabetes;
the cardiovascular risk of diabetes that is only partially explained by the conventional factors;
male erectile dysfunction;
pulmonary arterial hypertension;
cerebral hypoxia;
chronic rejection after organ transplantation;
cold ischaemia during organ transplantation;
extracorporeal circulation;
articular pathologies.

In the context of these pathologies, an ensemble of impairments representing cardiovascular risk factors has been combined under the term “syndrome X” or “metabolic insulin-resistance syndrome” (MIRS) (Reaven G M: Role of insulin resistance in human disease, Diabetes 1988; 37:1595-607); it includes insulin resistance, hyperinsulinism, glucose intolerance or diabetes, arterial hypertension and hypertriglyceridaemia.

Other anomalies are frequently associated with this syndrome: android obesity, microalbuminia, hyperglycaemia, clotting anomalies and fibrinolysis anomalies. Hepatic steatosis of non-alcoholic origin may also be associated therewith.

The administration of active ingredients capable of reducing the biological activity of oxidative free-radical species (such as superoxide anions and peroxy-nitrites) and of increasing the content of nitrogen monoxide by a twofold mechanism: non-conversion into peroxynitrites and exogenous supply, is thus particularly desirable in the treatment of these pathologies.

The present invention provides compounds that have both an antioxidant effect and a nitrogen monoxide-donating effect, which are capable of spontaneously generating nitrogen monoxide under physiological conditions and of trapping oxidative free radicals.

The spontaneous NO-donating effect does not induce a tachyphylactic effect, unlike compounds that are substrates of NO synthase, and unlike nitro derivatives or derivatives of oxadiazole or oxatriazole type which mobilise endogenous thiols groups to release NO.

Via the spontaneous NO-donating effect, pharmacological NO activity may be achieved in pathologies in which the activity of NO synthase is insufficient.

More specifically, the invention relates to the compounds of the formula I: 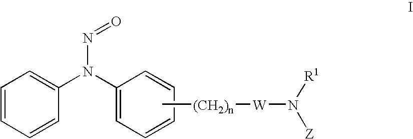
in which
each of the phenyl rings represented is optionally substituted one or more times;
n represents an integer selected from 0, 1, 2, 3, 4 and 5;
W represents —CO— or —SO₂—;
Z represents H; alkyl; aryl; or arylalkyl;
R₁ represents any monovalent organic group;
and the pharmaceutically acceptable salts thereof.

The expression “any monovalent organic substituent” is taken to mean any substituent attached to the —NZ- group via a carbon atom, and more particularly a substituent containing one or more carbon, nitrogen, oxygen, sulfur, phosphorus, halogen, silicon and hydrogen atoms.

Particularly preferred compounds are those of the formula Ia below: 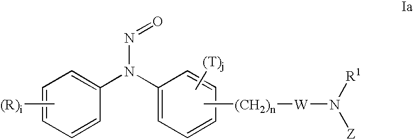
in which
W represents —CO— or SO₂—;
n represents an integer selected from 0, 1, 2, 3, 4 and 5;
i represents an integer selected from 0, 1, 2, 3, 4 and 5;
R₁ which may be identical or different, represent optionally halogenated alkoxy;
optionally halogenated alkylthio; optionally halogenated alkyl; optionally halogenated alkylsulfonyl; halogen; dialkylamino; cyano; alkylamino; or nitro;
Z represents H; alkyl; aryl; or arylalkyl;
T represents H or a halogen atom; or an alkyl group; an alkoxy group; an alkylthio group; an alkylamino group; or a dialkylamino group;
j represents an integer selected from 0, 1, 2, 3 and 4;
R₁ represents any monovalent organic group; and
the pharmaceutically acceptable salts thereof.

The expression “halogen atom” is taken to mean a fluorine, chlorine, bromine or iodine atom, preferably a chlorine or fluorine atom.

The expression “alkyl” is taken to mean a saturated hydrocarbon-based group containing a linear or branched chain, preferably having from 1 to 14 carbon atoms, preferably from 1 to 10 and better still from 1 to 6 carbon atoms, for example from 1 to 4 carbon atoms.

Examples of alkyl groups are methyl, ethyl, propyl, isopropyl, butyl, isobutyl, t-butyl, pentyl, isopentyl, neopentyl, 2-methylbutyl, 1-ethylpropyl, hexyl, isohexyl, neohexyl, 1-methylpentyl, 3-methylpentyl, 1,1-dimethylbutyl, 1,3-dimethylbutyl, 2-ethylbutyl, 1-methyl-1-ethylpropyl, heptyl, 1-methylhexyl, 1-propylbutyl, 4,4-dimethylpentyl, octyl, 1-methylheptyl, 2-methylhexyl, 5,5-dimethylhexyl, nonyl, decyl, 1-methylnonyl, 3,7-dimethyloctyl and 7,7-dimethyloctyl.

The expression “optionally interrupted with O and/or S” is taken to mean that any carbon atom not located at the free end of the hydrocarbon chain may be replaced by an oxygen or sulfur atom. The hydrocarbon chain, which may be alkyl, may contain plurality of oxygen and/or sulfur atoms, the hetero atoms preferably being separated from each other by at least one carbon atom and better still by at least two carbon atoms.

An example of an aliphatic hydrocarbon chain that is interrupted by O or S is alkoxy or thioalkoxy.

The aryl groups denote aromatic carbocyclic hydrocarbon-based groups, preferably of C₆-C₁₈. Among these, particular mention may be made of phenyl, naphthyl, anthryl and phenanthryl radicals.

The aryl groups are monocyclic or polycyclic; these radicals preferably denote monocyclic, bicyclic or tricyclic radicals. In the case of polycyclic radicals, it should be understood that they consist of monocycles fused in pairs (for example ortho-fused or peri-fused), i.e. containing in pairs at least two carbon atoms in common. Preferably, each monocycle is 3- to 8-membered and better still 5- to 7-membered.

The term “heteroaryl” is taken to mean a monocyclic or polycyclic, preferably monocyclic, bicyclic or tricyclic, aromatic heterocyclic group. In the case of polycyclic radicals, it should be understood that they consist of monocycles fused in pairs, i.e. containing in pairs at least two carbon atoms in common.

Each monocycle is preferably 3- to 8-membered and better still 5- to 7-membered. Each monocycle preferably contains from 1 to 4 hetero atoms and better still from 1 to 3 hetero atoms. These hetero atoms are selected from O, N and S, optionally in oxidised form (in the case of S and N).

Examples of monocyclic aromatic heterocyclic groups are 5- to 7-membered monocyclic heteroaryls, such as pyridine, furan, thiophene, pyrrole, pyrazole, imidazole, thiazole, isoxazole, isothiazole, furazane, pyridazine, pyrimidine, pyrazine, thiazines, oxazole, pyrazole, oxadiazole, triazole and thiadiazole.

Examples of bicyclic aromatic heterocyclic groups in which each monocycle is 5- to 7-membered are indolizine, indole, isoindole, benzofuran, benzopyran, benzothiophene, indazole, benzimidazole, benzothiazole, benzofurazane, benzothiofurazane, purine, quinoline, isoquinoline, cinnoline, phthalazine, quinazoline, quinoxaline, naphthyridine, pyrazolotriazine (such as pyrazolo-1,3,4-triazine), pyrazolopyrimidine and pteridine groups.

Examples of aromatic tricyclic heterocyclic groups are those consisting of 5- to 7-membered monocycles, such as acridine or carbazole.

The expression “any monovalent organic substituent (R¹)” is taken to mean any substituent attached to the —NZ- group via a carbon atom, and more particularly a substituent containing one or more carbon, nitrogen, oxygen, sulfur, phosphorus, halogen, silicon and hydrogen atoms.

Preferably, for the compounds of the formulae I and Ia, R¹ represents -A-Cy in which A represents a bond, alkylene or alkenylene; and Cy represents aryl, which is optionally substituted by one or more radicals St; heteroaryl, which is optionally substituted by one or more radicals St; or a saturated and/or unsaturated heterocycle, which is optionally substituted by one or more radicals St; or alternatively
R¹ represents -A-NRₐR_{b} in which A is as defined above; Rₐ represents H or alkyl; and R_{b} represents alkyl;
St is selected from nitro; a halogen atom; cyano; optionally halogenated alkylthio; alkylamino; dialkylamino; optionally halogenated alkyl; optionally halogenated alkoxy; a saturated and/or unsaturated heterocycle, which is optionally substituted by alkyl or alkoxy.

Even more preferably, R¹ represents optionally substituted phenyl; —(CH₂)ᵣ-Ph^{o} in which Ph^{o} is optionally substituted and r represents an integer selected from 1, 2 and 3, preferably 1; —B-phenyl in which B represents C₂-C₅ alkenylene; —(CH₂)ₜ-Het in which t is an integer selected from 0, 1, 2 and 3; and Het represents an optionally substituted saturated and/or unsaturated aromatic heterocycle, preferably monocyclic, containing 1 to 3 hetero atoms selected from N, O and S, or Het represents quinuclidine; —(CH₂)ₛ—NRₐR_{b} in which s is an integer selected from 0, 1 and 2 and Rₐ and R_{b}, which may be identical or different, are alkyl.

Advantageous meanings of —(CH₂)ₜ-Het are those in which Het represents a pyridyl; imidazolyl; piperidyl; piperazinyl; and pyrimidyl radical, the said radical optionally being substituted.

The saturated and/or unsaturated heterocyclic radicals include monocyclic and polycyclic radicals; these radicals preferably denote monocyclic, bicyclic or tricyclic radicals. Each monocycle is preferably 3- to 8-membered and better still 5- to 7-membered.

Each of the monocycles constituting the heterocycle preferably contains from 1 to 4 hetero atoms and better still from 1 to 3 hetero atoms. These hetero atoms are selected from O, N and S optionally in oxidised form. The polycyclic radicals are radicals in which each monocycle contains at least two carbon atoms in common with another monocycle. An example of a preferred tricyclic radical is quinuclidine.

The polycyclic radicals moreover comprise radicals consisting of monocycles fused in pairs (for example ortho-fused or peri-fused), i.e. containing at least two carbon atoms in common.

Examples of 7-membered unsaturated heterocycles include trithiatriazepines and trithiadiazepines. Examples of 5- to 7-membered saturated monocyclic heterocycles especially include tetrahydrofuran, dioxolane, imidazolidine, pyrazolidine, piperidine, dioxane, morpholine, dithiane, thiomorpholine, piperazine, trithiane, oxepine, azepine and pyrrolidine.

Examples of saturated and unsaturated bicyclic heterocyclic groups are the saturated or unsaturated derivatives of the aromatic heterocyclic groups mentioned above.

Similarly, examples of saturated or unsaturated tricyclic heterocyclic groups are the saturated or unsaturated derivatives of the tricyclic aromatic heterocyclic groups mentioned above.

The expression “saturated and/or unsaturated heterocyclic radicals” is taken to mean that the heterocyclic radical may comprise a saturated heterocyclic portion and/or an unsaturated heterocyclic portion.

According to the invention, the term “alkylene” represents a linear or branched divalent hydrocarbon-based chain having from 1 to 14 and preferably from 1 to 10 carbon atoms, better still from 1 to 6 carbon atoms, for example from 1 to 4 carbon atoms. Preferred examples of alkylene chains are methylene, ethylene and propylene chains.

An alkenylene chain is a linear or branched divalent hydrocarbon-based chain having from 2 to 14 carbon atoms, preferably from 2 to 10 carbon atoms and better still from 2 to 6 carbon atoms, for example from 2 to 4 carbon atoms, comprising one or more ethylenic unsaturations, for example from 1 to 3 ethylenic unsaturations. Examples of alkenylene chains are the chains:
—CH═CH—; —CH═C(CH₃)— and —CH₂—CH═CH—.

It should be understood that, according to the invention, the expression “saturated or unsaturated heterocycle” also includes the saturated and unsaturated heterocyclic monocyclic and polycyclic radicals defined above, fused to one or more aromatic carbocyclic (aryl) or aromatic heterocyclic (heteroaryl) rings, aryl and heteroaryl being as defined above. The fused aryl rings are preferably phenyl or naphthyl.

Similarly, the fused heteroaryl rings are pyridyl, quinolyl, benzofuryl, oxazolyl, thienyl, furyl, pyrrolyl, imidazolyl, thiazolyl, isoxazolyl, pyrazolyl, isothiazolyl, furazanyl, pyridazinyl, pyrimidinyl, pyridazinyl, pyrazinyl, thiazinyl, oxadiazolyl, triazolyl or thiadiazolyl.

A preferred subgroup of the compounds of the invention consists of the compounds of the formula I in which Z represents H.

Another preferred subgroup of the compounds of the invention consists of the compounds of the formula I in which W represents SO₂, R¹ represents —(CH₂)ₜ-Het in which t represents an integer selected from 0, 1, 2, 3 and 4 and Het represents an aromatic heterocycle, which is preferably monocyclic, containing 1 to 3 hetero atoms selected from O, N and S, the said heterocycle optionally being substituted.

Among these compounds, the ones that will be preferred are those for which Het represents pyridyl and t is 0 or 1.

A third group of compounds of the formula I consists of compounds for which W is —CO—; and R¹ represents —(CH₂)ₜ-Het in which t is an integer selected from 0, 1, 2 and 3; and Het represents an aromatic heterocycle, which is preferably monocyclic, containing 1 to 3 hetero atoms selected from O, N and S, the said heterocycle optionally being substituted.

Among these compounds, preference is given to those in which Het is pyridyl and t is 0 or 1.

A fifth group of compounds of the formula I consists of compounds for which the group —(CH₂)ₙ—W—N(Z)-R¹ is located in a meta position or in the para position relative to the —N—N═O group.

The invention also covers the salts that allow suitable separation or crystallisation of the compounds of the formula I, such as picric acid, oxalic acid or an optically active acid, for example tartaric acid, dibenzoyltartaric acid, mandelic acid or camphorsulfonic acid. However, a preferred subgroup of salts consists of the salts of the compounds of the formula I with pharmaceutically acceptable acids or bases.

Formula I includes all the types of geometrical isomers and stereoisomers of the compounds of the formula I.

Among the compounds that are more particularly preferred, mention will be made of:
4-[1-(4-methoxyphenyl)-2-oxohydrazino]-N-pyrid-3-ylbenzamide;
4-[1-(4-methoxyphenyl)-2-oxohydrazino]-N-pyrid-2-ylbenzamide;
4-[1-(4-methoxyphenyl)-2-oxohydrazino]-N-pyrid-3-ylmethylbenzamide.

The compounds of the invention can be prepared simply by nitrosation of the corresponding compounds of the formula II: 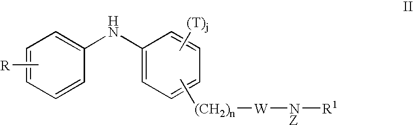
using a nitrosating agent, such as an alkali metal nitrite, in acidic medium.

Examples of nitrosating agents are alkali metal nitrites (and especially sodium or potassium nitrite) or a C₁-C₄ alkyl nitrite.

A preferred alkali metal nitrite that may be mentioned is sodium nitrite.

A preferred alkyl nitrite that may be mentioned is ethyl nitrite.

Nevertheless, a person skilled in the art can use any nitrosating agent known in the art, such as AgONO, BF₄NO, HOSO₃NO or nBuONO.

The amount of nitrosating agent required depends on the nature of the nitrosating agent used and on the reactivity of the substrate of the formula II. It is at least stoichiometric. In general, the molar ratio of the nitrosating agent to the substrate of the formula II ranges between 1 and 30 equivalents and preferably between 1 and 20 equivalents.

If the nitrosating agent is an alkali metal nitrite, a person skilled in the art may readily adapt the reaction conditions so as to use only 1 to 10, preferably from 1 to 5 and better still from 1 to 3 equivalents of nitrite relative to the substrate of the formula II.

If the nitrosating agent is an alkyl nitrite, it is preferable to carry out the process in the presence of from 10 to 25 molar equivalents of nitrite, and preferably from 15 to 20 molar equivalents, based on the amount of substrate of the formula II.

The choice of solvent and the temperature conditions depend especially on the type of nitrosating agent selected for the reaction.

If the nitrosating agent is AgONO, nBuONO or tBuONO, the solvent is advantageously selected from a cyclic or non-cyclic ether (such as diethyl ether, diisopropyl ether, tetrahydrofuran, dioxane, dimethoxyethane or diethylene glycol dimethyl ether), an aliphatic or aromatic halohydrocarbon (such as chloroform, carbon tetrachloride, dichloroethane, chlorobenzene or dichlorobenzene). The solvent is preferably tetrahydrofuran, diethyl ether or chloroform.

The reaction temperature will generally be maintained between 15 and 70° C. and better still between 17 and 60° C., in the case of AgONO, nBuONO and tBuONO.

More particularly, in the case of AgONO and nBuONO, the process will be carried out in tetrahydrofuran or diethyl ether at a temperature of between 15 and 30° C., for example between 18 and 25° C.

In the case of tBuONO, the process will preferably be carried out in chloroform at a temperature of between 40 and 65° C., for example between 50 and 60° C.

If the nitrosating agent is AgONO, it is desirable to add thionyl chloride to the reaction medium.

If the nitrosating agent is HOSO₃NO, the reaction is preferably carried out in an alkali metal salt of a lower (C₁-C₅) carboxylic acid, such as sodium acetate, at a reaction temperature of between −10° C. and 30° C. and better still between −5° C. and 25° C.

If the nitrosating agent is BF₄NO, a suitable solvent is a nitrile, such as acetonitrile or isobutyronitrile. It is desirable to add pyridine or N-dimethylaminopyridine to the reaction medium, the reaction temperature being maintained between −30° C. and 10° C. and preferably between −25° C. and 5° C.

If the nitrosating agent is an alkali metal nitrite, the nitrosation reaction is preferably carried out in a strongly polar protic medium. The reaction medium advantageously omprises water and a Brönsted or Lewis acid.

Suitable acids are a hydrohalic acid (such as HCl), sulfuric acid, Al₂(SO₄)₃ and acetic acid, and mixtures thereof.

According to one particular embodiment of the invention, an aliphatic alcohol of (C₁-C₄)alkanol type (such as methanol or butanol) may be added.

Thus, a suitable reaction medium that may be selected is one of the following systems:
a mixture of methanol, water, hydrochloric acid and sulfuric acid;
a mixture of water and sulfuric acid;
a mixture of water and acetic acid;
a mixture of water, butanol and hydrochloric acid;
a mixture of water and Al₂(SO₄)₃, or
a mixture of water and hydrochloric acid.

The reaction of the alkali metal nitrite with the substrate of the formula II is advantageously carried out in a mixture of acetic acid and water, the ratio of the acetic acid to water ranging between 80:20 and 20:80 and preferably between 60:40 and 40:60, for example a 50:50 mixture. According to one preferred embodiment, the alkali metal nitrite, pre-dissolved in water, is added dropwise to a solution of the substrate of the formula II in acetic acid.

The reaction of the alkali metal nitrite with the substrate of the formula II is carried out at a temperature which depends on the reactivity of the species present; this temperature generally ranges between −10° C. and 50° C. and preferably between −5° C. and 25° C.

If the nitrosation reaction is carried out in a mixture of acetic acid and water, a temperature of between 15° C. and 25° C. is particularly suitable.

The reaction of the alkyl nitrite with the substrate of the formula II is preferably carried out in the presence of a C₁-C₄ alkanol in a polar aprotic solvent.

Suitable alkanols that may be mentioned include methanol, ethanol, isopropanol and tert-butanol, ethanol being particularly preferred.

Preferred polar solvents are halohydrocarbons, such as methylene chloride, chloroform, carbon tetrachloride, dichloroethane, chlorobenzene or dichlorobenzene; ethers, such as diethyl ether, diisopropyl ether, tetrahydrofuran, dioxane, dimethoxyethane or diethylene glycol dimethyl ether; nitriles, such as acetonitrile or isobutyronitrile; amides, such as formamide, dimethylformamide, dimethylacetamide, N-methyl-2-pyrrolidinone or hexamethylphosphoramide; and mixtures of these solvents in any proportions.

The nitrosation reaction (if an alkyl nitrite is used as nitrosating agent) is advantageously carried out in a mixture based on an aliphatic halohydrocarbon and a nitrile, and for example in a 90:10 to 50:50 and preferably a 90:10 to 70:30 mixture of chloroform and acetonitrile, in the presence of ethanol.

The amount of alkanol that needs to be incorporated into the reaction, medium is not critical in accordance with the invention. It generally represents 5% to 50% by weight of the reaction medium, and preferably from 5% to 25% by weight.

If the nitrosating agent is an alkyl nitrite, the reaction temperature is generally maintained between −20° C. and 20° C. and preferably between −10° C. and 10° C., for example between 0° C. and 5° C.

According to one preferred embodiment of the invention, a solution of the alkyl nitrite in the alkanol is added dropwise to the substrate of the formula II predissolved in the selected polar solvent.

As a variant, the reaction is carried out in a strongly polar medium consisting of a mixture of a C₁-C₄ aliphatic carboxylic acid ((C₁-C₄)alkyl-COOH), the corresponding acid anhydride and the corresponding alkali metal carboxylate salt, in the presence of P₂O₅. By way of example, a reaction medium consisting of acetic acid, acetic anhydride, potassium acetate and P₂O₅ can be selected. In this case, the reaction temperature is advantageously maintained between 10° C. and 100° C. and preferably between 15° C. and 85° C.

The compounds of the formula II can be prepared by carrying out one of the following processes.

A—Preparation of the Compounds of the Formula II in which W Represents CO or SO₂.

One method for preparing the compounds of the formula II in which W represents CO or SO₂ consists in reacting a compound of the formula V: 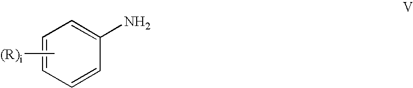
in which
R and i are as defined above for formula II, with a compound of the formula VI: 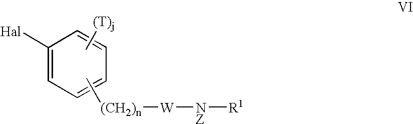
   in which
Hal represents a halogen atom, such as bromine or chlorine, preferably bromine;
T, j, n, W, Z and R¹ are as defined above.

This reaction is advantageously carried out in the presence of a base. Examples of bases that may be selected are any one of those mentioned above. Preferably, an alkali metal alkoxide, such as sodium or potassium methoxide, ethoxide or tert-butoxide will be selected, and will be introduced into the reaction medium in a proportion of 1 to 2 equivalents per one equivalent of compound VI, for example between 1.2 and 1.7 equivalents.

This reaction is generally carried out at a temperature of between 50 and 180° C. and preferably at a temperature of between 80 and 150° C.

The temperature depends on the nature of the species present and especially the strength of the base and the reactivity of the compounds V and VI present.

The solvent is generally selected from the polar aprotic solvents defined above.

Preferred solvents that may be mentioned include ethers and especially glymes, such as 1,2-dimethoxyethane, diethylene glycol dimethyl ether (diglyme) or triethylene glycol dimethyl ether (triglyme), diglyme being more particularly preferred, and aromatic hydrocarbons, such as xylene and toluene.

According to one preferred embodiment of the invention, the molar ratio of the amine V to the compound VI ranges between 1 and 2 and better still between 1 and 1.5, for example between 1.1 and 1.3.

Advantageously, it is desirable to introduce a palladium(0) catalyst into the reaction medium.

Acatalyst of this type can be obtained by introducing into the reaction medium the system (dba)₃Pd₂ (tris(dibenzylideneacetone)dipalladium(0))+BINAP, in which BINAP is the diphosphine of the formula:

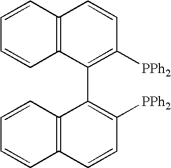

By way of illustration, each of the catalytic substances (dba)₃Pd₂ and BINAP is introduced into the reaction medium in a proportion of less than 10% by weight. In a particularly advantageous manner, the molar ratio of the BINAP to the (dba)₃Pd₂ ranges between 1.5 and 4 and preferably between 2 and 3.

A person skilled in the art may be inspired to carry out this reaction by J. Org. Chem. (2000), 65, 1144-1157.

B. Preparation of the Compounds of the Formula II in which W Represents CO.

One method for preparing the compounds of the formula II in which W represents CO consists in successively carrying out the following steps:
i) a compound of the formula VII: 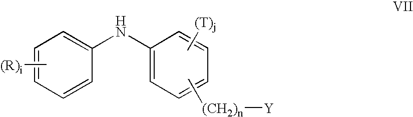
   in which:
R, i, T, j and n are as defined for formula II and Y represents an ester function, such as alkoxycarbonyl; aryloxycarbonyl; arylalkoxycarbonyl; in which the aryl and alkyl portions are as defined above and are optionally substituted by alkyl, alkoxy or halogen, is reacted with a suitable electrophilic agent so as to protect the amine function of the diphenylamine of the formula VII above; by means of which a compound of the formula VII is isolated: 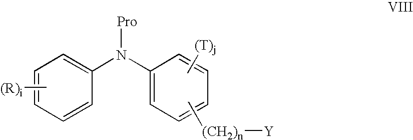
   in which:
R, i, T, j, n and Y are as defined above and Pro represents a protecting group,
ii) the ester function of the resulting compound of the formula VIII is saponified, using a suitable base, which gives the carboxylic acid of the formula: 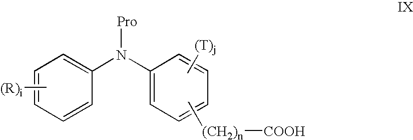
   in which
R, i, T, j, Pro and n are as defined above;
iii) the carboxylic acid of the formula IX is coupled with an amine of the formula X:
R¹—NZH, optionally after activation of the carboxylic function; which gives the compound of the formula XII: 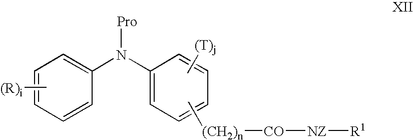
   in which
R, i, Pro, T, j, n, Z and R¹ are as defined above;
iv) the protecting function Pro is removed so as to release the amine function of the diphenylamine, by means of which the compound of the formula II is isolated.

In step i), a person skilled in the art may select any of the protecting groups known in the art, which are described especially in “Protective Groups in Organic Synthesis”, Greene T. W. and Wuts P. G. M., published by John Wiley & Sons, 1991, and “Protecting Groups”, Kocienski P. J., 1994, Georges Thieme Verlag.

By way of example, the amine function may be protected by a tert-butoxy-carbonyl function.

With this aim, the compound of the formula VII may be reacted with at least one equivalent of di-tert-butyl dicarbonate, in the presence of a strong base, such as an ammonium or alkali metal hydroxide or in the presence of an alkali metal hydride, such as sodium hydride.

This reaction is preferably carried out in a polar aprotic solvent, such as an optionally halogenated aromatic or aliphatic hydrocarbon; an ether (diethyl ether, diisopropyl ether, tetrahydrofuran, dioxane, dimethoxyethane or diethylene glycol dimethyl ether); a ketone (acetone, methyl ethyl ketone, isophorone or cyclo-hexanone); a nitrile (acetonitrile or isobutyronitrile); an amide (formamide, dimethylformamide, dimethylacetamide, N-methyl-2-pyrrolidinone or hexamethyl-phosphorylamide).

Dimethylformamide is preferably selected as solvent.

The reaction temperature is preferably between 0 and 35° C., for example between 5 and 25° C.

Other groups for protecting the amino function, acyl groups of the type R—CO (in which R is a hydrogen atom or an alkyl, cycloalkyl, aryl, arylalkyl or heteroarylalkyl radical, R optionally being substituted by alkyl, alkoxy or halogen), urea-forming groups of the formula —CO—NA₂B₂ or urethane-forming groups of the formula —CO—OA₂ (in which A₂ and B₂ are, independently, alkyl, aryl, arylalkyl or cycloalkyl—optionally substituted by alkyl, alkoxy or halogen—or alternatively A₂ and B₂, together with the nitrogen atom that bears them, form a monocyclic or polycyclic, preferably monocyclic or bicyclic, saturated, unsaturated or aromatic heterocycle, which is optionally substituted by alkyl, alkoxy or halogen), thio-urethane-forming groups of the formula —CS—NA₂B₂ (in which A₂ and B₂ are as defined above), diacyl groups in which: 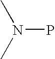
in formulae III and IV represents the group: 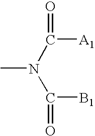
in which:
A₁ and B₁ are, independently, alkyl, aryl, arylalkyl or cycloalkyl—optionally substituted by alkyl, alkoxy or halogen—or alternatively A₁ and B₁ form, together with N and the two carbonyl groups, a monocyclic or polycyclic, preferably monocyclic or bicyclic, saturated, unsaturated or aromatic heterocycle optionally substituted by alkyl, alkoxy or halogen—such as phthalimide, tetrahydropyranyl groups, and less commonly alkyl groups, alkenyl groups (allyl or isopropenyl), arylalkyl groups, such as trityl or benzyl, and groups of benzylidene type.

Examples of amino protecting groups that may be mentioned are the formyl group, the acetyl group, the chloroacetyl group, the dichloroacetyl group, the phenylacetyl group, the thienylacetyl group, the tert-butoxycarbonyl group, the benzyloxycarbonyl group, the trityl group, the p-methoxybenzyl group, the diphenylmethyl group, the benzylidene group, the p-nitrobenzylidene group, the m-nitrobenzylidene group, the 3,4-methylenedioxybenzylidene group and the m-chlorobenzylidene group.

Particularly preferred protecting groups especially (C₁-C₆)alkoxycarbonyl and (C₈-C₁₀)aryl-(C₁-C₆)alkoxycarbonyl, such as tert-butoxycarbonyl and benzyloxycarbonyl.

In step ii), the ester function is saponified. The saponification is carried out in a manner known per se in the presence of a strong base, generally a mineral base selected from NaOH, KOH, NaHCO₃, Na₂CO₃, KHCO₃ and K₂CO₃.

The saponification can be carried out in a mixture of water and a lower alcohol, such as ethanol or methanol. The process is advantageously carried out in the presence of an excess of base relative to the amount of ester of the formula VIII. By way of example, the molar ratio of the base to the compound of the formula VIII ranges between 1 and 5 equivalents and preferably between 1 and 3 equivalents.

In step iii), the coupling is preferably carried out by reacting the amine R¹—NHZ with an activated form of the said acid, optionally prepared in situ.

Activating groups that are preferred for the carboxylic acid function, which are well known in the prior art, are, for example, chlorine, bromine, an azide, imidazolide, p-nitrophenoxy or 1-benzotriazole group, an N—O-succinimide group, acyloxy and more particularly pivaloyloxy, (C₁-C₄ alkoxy)carbonyloxy, such as C₂H₅O—CO—O—, dialkyl- or dicycloalkyl-O-ureide.

The reaction of the amine X, of the formula R¹—NHZ, with the carboxylic acid of the formula XII, optionally in activated form, is preferably carried out in the presence of a coupling agent, such as a carbodiimide or bis(2-oxo-3-oxazolidinyl)phosphonyl chloride. Examples of carbodiimides are especially dicyclohexyl- and diisopropylcarbodiimides or carbodiimides that are soluble in an aqueous medium. Another type of coupling agent is oxalyl chloride.

The process is advantageously carried out in the presence of a base, such as an organic base. Preferred examples of bases are triethylamine, tributylamine and diisopropylethylamine.

The process is generally carried out in a polar aprotic solvent, such as one of those mentioned above.

Optionally halogenated aliphatic and aromatic hydrocarbons that may be mentioned include benzene, toluene, xylene, methylene chloride, chloroform, carbon tetrachloride, dichloroethane, chlorobenzene and dichlorobenzene.

Among the preferred solvents that will primarily be selected are a glyme, such as diglyme, dimethylformamide and methylene chloride, and mixtures thereof.

The amount of coupling agent is preferably at least equal (in molar percentages) to the amount of acid of the formula IX. Preferably, the molar ratio of the coupling agent to the acid of the formula IX ranges between 1 and 3 equivalents, for example between 1 and 2.

As regards the molar ratio of the base to the acid, this preferably ranges between 1 and 3 equivalents, preferably between 1 and 2 equivalents.

The coupling agents that are preferred are oxalyl chloride and bis(2-oxo-3-oxazolidinyl)phosphonyl chloride.

A preferred base that will be mentioned is triethylamine.

The procedure generally followed involves reacting the acid with the coupling agent, optionally in the presence of the base, at a temperature that ranges between 15° C. and 55° C., for example between room temperature and 45° C.

In a second stage, the amine of the formula X is introduced into the reaction medium optionally in combination with the base selected for the reaction, and the mixture is brought to a temperature of between 80° C. and 150° C., for example between 110° C. and 130° C.

The preparation of the compounds of the formula II in which W represents CO may be carried out without intermediate protection of the nitrogenous function of the diphenylamine, by carrying out:
a first saponification step similar to step ii) described above, but using as starting material the compound of the formula VII in unmodified form;
a second step of coupling the resulting carboxylic compound of the formula XVII as defined above with an amine of the formula X: R¹—NZH as defined above under reaction conditions similar to those generally described above in step iii).

The compounds of the formula VII may be obtained by reacting a compound of the formula XI: 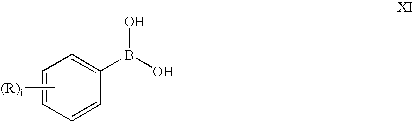
in which:
R and i are as defined above, with a compound of the formula XIII 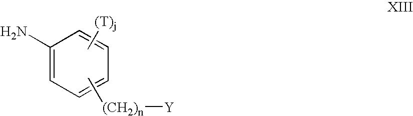
   in which:
n, T, j and Y are as defined above, in the presence of a suitable activator, such as copper acetate, and of a base, preferably an organic base.

Advantageously, the molar ratio of compound XI to compound XIII ranges between 1 and 5 equivalents and preferably between 1.2 and 3, for example between 1.5 and 2.5.

The base is preferably used in a proportion of from 1 to 5 molar equivalents relative to the amount of compound XIII.

Finally, about 1 to about 2 equivalents of copper acetate relative to the amount of compound XIII are generally used.

The reaction is preferably carried out in a polar aprotic solvent as defined above, for example dichloromethane, at room temperature, i.e. at a temperature of between 15 and 30° C.

As a variant, the compounds of the formula VII may be prepared by reacting an amine XIV: 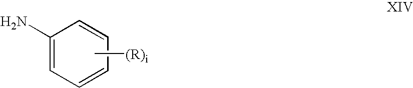
in which:
R and i are as defined above, with a compound of the formula XV: 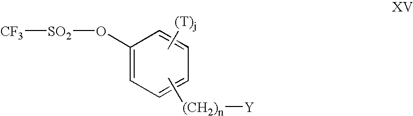
   In which T, j, n and Y are as defined above, in the presence of Cs₂CO₃ and a mixture of Pd(OAc)₂ and BINAP, BINAP corresponding to the formula:

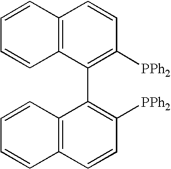

According to one preferred embodiment of the invention, the molar ratio of compound XIV to compound XV ranges between 1 and 3 equivalents and preferably between 1 and 2 equivalents.

Cs₂CO₃ is used in a proportion of from 1 to 2 equivalents, for example 1 to 1.5 equivalents, relative to the amount of compound XV.

Pd(OAc)₂ and BINAP are used in catalytic amount.

The reaction is carried out in a polar aprotic organic solvent, such as an aromatic hydrocarbon, for example in toluene, at a temperature of between 40 and 150° C., for example between 80 and 110° C.

Another preparation variant for the compounds of the formula VII consists in reacting an amine of the formula XIV, as defined above, with a compound of the formula XVI: 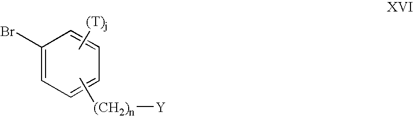
in which T, j, n and Y are as defined above, in the presence of a mixture of Pd(dba)₂ and P(tBu)₃ and of a base of alkali metal alkoxide type, such as potassium or sodium methoxide, ethoxide or tert-butoxide.

Pd(dba)₂ denotes bis(dibenzylideneacetone)palladium.

This reaction is preferably carried out in an apolar aprotic solvent, such as an aromatic hydrocarbon, for example toluene.

The molar ratio of compound XVI to compound XIV generally ranges between 1 and 1.5 equivalents, whereas Pd(dba)₂ and P(tBu)₃ are used in catalytic amount.

The base is generally incorporated into the reaction medium in a large excess.

The compound of the formula IX can be obtained by introducing a protecting group for the amino function, starting with a compound of the formula XVII: 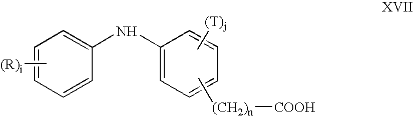
in which:
R, i, j, T and n are as defined above.

For the determination of the reaction conditions, a person skilled in the art may be inspired by the conditions generally described above in method B, step i for the preparation of the compounds of the formula II in which W represents CO.

The compounds of the formula XVII can be obtained simply from the corresponding compounds of the formula XVIII: 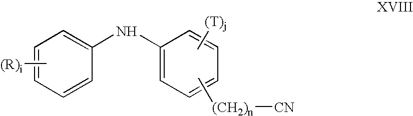
in which:
R, i, T, j and n are as defined above, by the action of a base, such as a mineral base. Among the mineral bases mentioned above, KOH and NaOH are preferred.

This reaction is generally carried out in a solvent, such as an aqueous solvent, or in alcoholic medium (for example in a lower alcohol, such as methanol or ethanol, the term “lower” denoting alcohols containing from 1 to 6 carbon atoms).

Another type of solvent consists of ethers, such as ethylene glycol, propylene glycol and polyethylene glycol. The reaction temperature ranges from room temperature (15-25° C.) to 150° C.

The compounds of the formula XVIII can be prepared by coupling a compound of the formula XIV as defined above with a compound of the formula XIX: 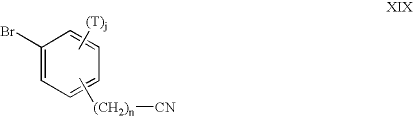
in which:
T, j, and n are as defined above, in the presence of a base of alkali metal alkoxide type and a mixture of Pd(dba)₂ and P(tBu)₃.

The conditions for carrying out this reaction are of the type recommended in the case of the reaction of compound XIV with compound XVI.

The compounds of the formula XVIII can also be prepared by the coupling reaction of an amine of the formula XIV with a compound of the formula XX: 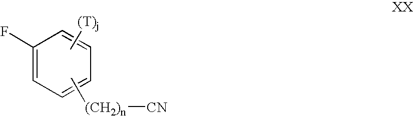
In which T, j and n are as defined above, in the presence of a base of alkali metal alkoxide type, preferably potassium tert-butoxide.

Suitable solvents are especially polar solvents and more particularly solvents of the amide or nitrile type, such as acetonitrile or isobutyronitrile, formamide, dimethylformamide, dimethylacetamide or hexamethylphosphorylamide; or alternatively a solvent of the type such as dimethyl sulfoxide.

The process is preferably carried out in the presence of an equimolar amount of compounds XIV and XX. However, it may be advantageous to carry out the process in the presence of an excess of amine XIV, for example up to 5 equivalents and better still up to 2 equivalents.

This reaction is advantageously carried out in dimethyl sulfoxide, the molar ratio of the base to the compound of the formula XX ranging between 1 and 5 equivalents and preferably between 1 and 3 equivalents.

The invention also relates to the compounds of the formula II that are novel.

Among these compounds that are distinguished more particularly are the compounds of the formula III: 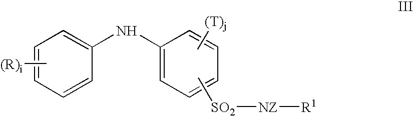
in which:
i, j, R, Z and T are as defined above;
R¹ represents phenyl, which is optionally substituted by one or more radicals St;
—(CH₂)ᵣ-Ph^{o}, in which Ph^{o} is optionally substituted by one or more radicals St and r represents an integer selected from 1, 2 and 3, or alternatively R¹ represents —(CH₂)ₜ-Het, in which Het is a radical selected from pyridyl; imidazolyl; piperidyl; piperazinyl; and pyrimidyl, the said radical being optionally substituted by one or more radicals St, and t is selected from an integer 0, 1, 2 and 3; with the exclusion of the following compounds defined by the formula III in which:
a) R in position 2=R in position 4=NO₂; i=2; j=0; Z=H; and R¹=2-pyridyl; or
b) R in position 2=R in position 4=NO₂; i=2; j=0; Z=H; and R¹ represents 2,6-dimethyl-4-pyrimidyl, or 4,6-dimethyl-2-pyrimidyl;
c) R¹ represents phenyl; Z=H; i=0,1; j=0; and R represents diethylamino;
d) R¹ represents 2,4-dinitrophenyl; i=2; R in position 2=R in position 4=NO₂; j=0; Z=H;
e) R¹ represents 2,4,6-triisopropylphenyl; Z=H; i=1; j=0; R=di(n-hexyl)amino;
f) R in position 2=R in position 6=R in position 4=NO₂; i=3; j=0; Z=H; R¹=2,6-dimethoxy-4-pyrimidinyl.

Other preferred compounds of the formula II are the compounds of the formula III in which:
i, j, R, Z and T are as defined above;
W═—CO—;
R¹ represents phenyl, which is optionally substituted by one or more radicals St;
—(CH₂)ᵣ-Ph^{o}, in which Ph^{o} is optionally substituted by one or more radicals St and r represents an integer selected from 1, 2 and 3; or R¹ represents —(CH₂)ₜ-Het, in which Het is a radical selected from pyridyl; imidazolyl; piperidyl; piperazinyl; and pyrimidyl, the said radical being optionally substituted by one or more radicals St, St is selected from nitro; a-halogen atom; cyano; optionally halogenated alkylthio; alkylamino; dialkylamino; optionally halogenated alkyl; optionally halogenated alkoxy; a saturated or unsaturated heterocycle optionally substituted by alkyl or alkoxy, and t is selected from an integer 0, 1, 2 and 3; with the exclusion of the following compounds defined by formula ill in which:
a) R₁=4-methyl-3-nitrophenyl; 4-ethoxyphenyl; 2-bromo-4-nitrophenyl; phenyl; 4-bromophenyl; 2-chlorophenyl; 3-fluorophenyl; 4-methoxyphenyl; 2-methoxyphenyl; 4-dimethylaminophenyl; 3-methoxyphenyl; 2,4-dinitrophenyl; 4-methylphenyl; 3-methylphenyl; or 2-methylphenyl; i=2, 3; R=NO₂; j=0;
b) R₁=2-pyridyl; i=3; R=NO₂; j=0;

Other compounds of the formula II that are distinguished are the compounds of the formula IV: 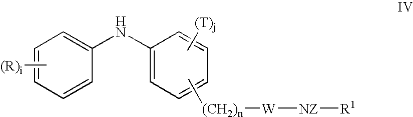
in which:
W represents —CO— or —SO₂—;
i, j, R, Z and T are as defined in claim 1;
R¹ represents phenyl, which is optionally substituted by one or more radicals St; —(CH₂)ᵣ-Ph^{o}, in which Ph^{o} is optionally substituted by one or more radicals St, St is selected from nitro; a halogen atom; cyano; optionally halogenated alkylthio; alkylamino; dialkylamino; optionally halogenated alkyl; optionally halogenated alkoxy; a saturated or unsaturated heterocycle, which is optionally substituted by alkyl or alkoxy, and r represents an integer selected from 1, 2 and 3; or R¹ represents —(CH₂)ₜ-Het, in which Het is a radical selected from pyridyl; imidazolyl; piperidyl; piperazinyl; and pyrimidyl, the said radical being optionally substituted is by one or more radicals St selected from nitro; a halogen atom; cyano; optionally halogenated alkylthio; alkylamino; dialkylamino; optionally halogenated alkyl; optionally halogenated alkoxy; a saturated and/or unsaturated heterocycle, which is optionally substituted by alkyl or alkoxy; and t is selected from the integers 0, 1, 2 and 3.

The compounds of the formula II above can be used not only as intermediates in the synthesis of the compounds of the formula I, but also have an antioxidant activity that makes them capable of limiting the destructive activity of oxidative free-radical species.

The compounds of the formula I of the invention increase the level of nitric oxide.

A solution of a compound of the formula I of the invention spontaneously releases nitric oxide. The nitrite ions resulting therefrom are titrated by colorimetry by means of a specific reagent (Griess). To take account of any release of nitrate ions in addition to the nitrites, bacterial nitrate reductase is added to the reaction medium to reduce the nitrate ions formed.

The following tests were carried out so as to demonstrate this activity.

The reactions and measurements are carried out in transparent 96-well plates. The test products are dissolved at the time of use, at a concentration of 3 mM in dimethyl sulfoxide. 95 μl of a reagent containing nitrate reductase (0.18 U/ml in 100 mM pH 7.5 PBS buffer, 210 μM β-NADPH, 5 μM FAD) and 5 μl of the solution of the test product (final concentration of 150 μM) are then added to each well. After stirring, the mixtures are incubated for 4 hours at 37° C. The reaction is then stopped by adding 100 μl of Griess' reagent (Sigma G4410). The resulting mixture is stirred for 5 min at room temperature, and the optical density is then read at 540 nm. This value is proportional to the concentration of nitrites+nitrates in the medium. A calibration range is made for each plate, using NaNO₂.

The results are expressed as μmol/l (μM) of nitrites+nitrates released in Table A for some of the compounds of the formula I given as examples below.

The compounds of the formula I of the invention decrease the biological activity of oxidative free-radical species.

The protocol used to demonstrate the activity of the compounds of the formula I is described below.

Human LDLs placed in aqueous solution in the presence of cupric ions, become spontaneously oxidised on their protein component, apolipoprotein-B. This oxidation makes the particle fluorescent, which is exploited to measure a pharmacological effect.

The reactions and measurements are carried out in black 96-well plates. 10 μl of a solution of the test product dissolved in dimethyl sulfoxide are first mixed with 170 μl of a solution of human LDL at a concentration of 120 μg/ml and 20 μl of 100 μM CuCl₂. After stirring, the mixture is incubated for 2 hours at 37° C., and a first fluorescence reading is taken (excitation at 360 nm, reading at 460 nm). The mixture is then incubated for a further 22 hours, to take a second reading under the same conditions. The difference is proportionately smaller the greater the antioxidant power of the test product. Probucol is used as reference product at a concentration of 10 μM.

The concentrations that inhibit 50% (IC₅₀) of the oxidation are prepared from three concentrations of the test product. They are given in Table B below for some of the compounds of the formula I given as examples below.

TABLE A

| | Nitrites-Nitrates |
|---|---|
| Examples | (μM) |
| 1b | 63 |
| 4b | 70 |
| 5b | 47 |
| 6b | 58 |
| 9b | 46 |
| 10b | 67 |
| 13b | 92 |
| 14b | 90 |
| 15b | 82 |
| 16b | 97 |
| 17b | 82 |
| 18b | 81 |
| 19b | 52 |
| 20b | 53 |
| 22b | 68 |
| 23b | 60 |
| 29b | 90 |
| 54b | 108 |
| 55b | 60 |
| 58b | 96 |
| 132b | 82 |
| 133b | 51 |
| 134b | 55 |
| 135b | 75 |
| 136b | 98 |
| 137b | 94 |
| 138b | 95 |
| 139b | 88 |

TABLE B

| | Antioxidant effect IC₅₀ |
|---|---|
| Examples | (μM) |
| 1b | 4.6 |
| 4b | 12.7 |
| 15b | 4.8 |
| 132b | 9.3 |
| 135b | 8.6 |

The compounds of the formula II above can be used not only as intermediates in the synthesis of the compounds of the formula I, but also have an antioxidant activity that makes them capable of limiting the destructive activity of oxidative free-radical species.

The antioxidant activity of the compounds of the formula II is revealed in vitro, for example, by evaluating the ability of the compounds of the formula II to prevent the oxidation of low molecular weight human lipoproteins.

The IC₅₀ values measured in the case of a certain number of compounds of the formula II are given in Table C below.

TABLE C

| | Antioxidant effect IC₅₀ |
|---|---|
| Examples | (μM) |
| 4a | 9.1 |
| 8a | 10.0 |
| 33a | 12.9 |
| 132a | 7.7 |
| 134a | 4.5 |
| 135a | 7.5 |
| 136a | 19.8 |

The compounds of the invention of the formulae I and II also have a hypo-triglyceridaemiant activity. This activity was especially observed by the inventors on a pathological animal model.

The compounds of the formulae I and II of the invention moreover have the effect of reducing the levels of free fatty acids in the blood and of increasing the levels of HDL cholesterol in the blood.

The effect of the treatment has an impact on insulinaemia, which is lowered, and allows insulin resistance to be modulated.

These properties of the compounds of the invention are useful in the prevention and treatment of diabetes, especially on account of the improvement in the sensitivity to insulin.

Thus, according to another of its aspects, the invention relates to the use of the compounds of the formulae I and II of the invention for the preparation of a medicament that can be used in the treatment of metabolic insulin resistance syndrome (MIRS).

According to another of its aspects, the invention relates to a pharmaceutical composition comprising at least one compound of the formula I as defined above in combination with at least one pharmaceutically acceptable excipient.

According to yet another of its aspects, the invention relates to a pharmaceutical composition comprising at least one compound of the formula II in combination with at least one pharmaceutically acceptable excipient.

These compounds can be administered orally in the form of tablets, gel capsules or granules with immediate release or controlled release, intravenously in the form of an injectable solution, transdermally in the form of an adhesive transdermal device, or locally in the form of a solution, cream or gel.

A solid composition for oral administration is prepared by adding to the active ingredient a filler and, where appropriate, a binder, a disintegrant, a lubricant, a colorant or a flavour corrector, and by shaping the mixture into a tablet, a coated tablet, a granule, a powder or a capsule.

Examples of fillers include lactose, corn starch, sucrose, glucose, sorbitol, crystalline cellulose and silicon dioxide, and examples of binders include poly(vinyl alcohol), poly(vinyl ether), ethylcellulose, methylcellulose, acacia, gum tragacanth, gelatine, shellac, hydroxypropylcellulose, hydroxypropylmethyl-cellulose, calcium citrate, dextrin and pectin. Examples of lubricants include magnesium stearate, talc, polyethylene glycol, silica and hardened plant oils. The colorant may be any colorant permitted for use in medicaments. Examples of flavour correctors include cocoa powder, mint in herb form, aromatic powder, mint in oil form, borneol and cinnamon powder. Needless to say, the tablet or granulate may be suitably coated with sugar, gelatine or the like.

An injectable form comprising the compound of the present invention as active ingredient is prepared, where appropriate, by mixing the said compound with a pH regulator, a buffer agent, a suspending agent, solubiliser, a stabiliser, a tonicity agent and/or a preservative, and by converting the mixture into a form for intravenous, subcutaneous or intramuscular injection, according to a conventional process. Where appropriate, the injectable form obtained may be freeze-dried by a conventional process.

Examples of suspending agents include methylcellulose, polysorbate 80, hydroxyethylcellulose, acacia, powdered gum tragacanth, sodium carboxymethyl-cellulose and polyethoxylated sorbitan monolaurate.

Examples of solubilisers include castor oil solidified with polyoxyethylene, polysorbate 80, nicotinamide, polyethoxylated sorbitan monolaurate and the ethyl ester of castor oil fatty acid.

In addition, the stabiliser encompasses sodium sulfite, sodium metasulfite and ether, while the preserving agent encompasses methyl p-hydroxybenzoate, ethyl p-hydroxybenzoate, sorbic acid, phenol, cresol and chlorocresol.

According to another of its aspects, the invention relates to the use of a compound of the formula I as defined above for the preparation of a medicament for treating pathologies that are characterised by a lack of nitrogen monoxide production and/or an oxidative stress condition.

According to one of its final aspects, the invention relates to the use of a compound of the formula II for the preparation of an antioxidant medicament that can be used as a free-radical scavenger.

The present invention is illustrated below in the light of the following examples.

The frequency of the NMR machine used to record the proton spectra in the examples given below is 300 MHz.

The LC-MS spectra are obtained on a simple quadrupole machine, equipped with an electrospray probe.

### EXAMPLE 1

### 4-[1-(4-Methoxyphenyl)-2-oxohydrazino]-N-pyrid-3-ylbenzamide

### a) 4-[(4-Methoxyphenyl)amino]-N-pyrid-3ylbenzamide

5.4 g (5.85 mmol) of tris(dibenzylideneacetone)dipalladium (0), 10.9 g (17.55 mmol) of racemic BINAP (2,2-bis(diphenylphosphino)-1,1-binaphthyl) and 33.7 g (351 mmol) of sodium t-butoxide are added to a mixture, under nitrogen, of 65 g (234 mmol) of 4-bromo-N-pyrid-3-ylbenzamide prepared according to C.A. (1967), 66, 37125 h, 34.7 g (281 mmol) of 4-methoxyaniline and 825 ml of diglyme (diethylene glycol dimethyl ether). The reaction mixture is heated at 130° C. for 15 hours. After cooling, 4 l of water are added and the mixture is extracted with ethyl acetate.

The organic phase is washed with H₂O, dried over Na₂SO₄ and then concentrated to give a solid residue, which, after trituration in 250 ml of dichloro-methane and drying under vacuum, is recrystallised from ethanol to give 41.8 g of a beige-coloured solid.

(Yield=55.9%).

m.p.=178-180° C.

IR (KBr): ν=3235 (NH); 1647 (CO)

LC-MS ES⁺: 320.34 (M+1)

NMR (DMSO-d₆): 3.84 and 3.86 (3H, 2s); 6.9 (4H, m); 7.2 (2H, m); 7.4 (1H, m); 7.85 (2H, d, J=8.7 Hz); 8.2 (1H, m); 8.3 (1H, m); 8.5 (1H, s); 8.9 (1H, d, J=2.2 Hz); 10.1 (1H, s, exchangeable with CF₃COOD).

### b) 4-[1-(4-Methoxyphenyl)-2-oxohydrazino]-N-pyrid-3-ylbenzamide

A solution of 18.1 g (262 mmol) of sodium nitrite in 375 ml of water is added dropwise, at room temperature, to a solution of 41.8 g (131 mmol) of the compound prepared in Example 1a, in 1300 ml of acetic acid.

After stirring for 2.5 hours at room temperature, the reaction medium is poured into 8.7 l of ice-cold water and extracted with CHCl₃ (3×1 l) and then with CH₂Cl₂ (6 l).

The organic phase, separated out after settling of the phases, is washed with NaHCO₃ solution and then with water until neutral, after which it is dried over Na₂SO₄.

After filtration and concentrating under vacuum at 25° C., a solid is obtained, which is triturated with 600 ml of pentane.

The solid is filtered off and dried under vacuum at room temperature to give 44.2 g of an orange-beige solid.

(Yield: 96.9%).

m.p.=167-169° C.

IR (KBr): ν=3326 (NH); 1649 (CO)

LC-MS ES⁻: 347.29 (M−1)

LC-MS ES⁺: 319.30 (M−NO+1)

NMR (DMSO-d₆) of the 2 conformers: 3.80 (3H, 2s); 7.0-7.6 (7H, m); 8.05 (2H, m); 8.15 (1H, m); 8.30 (1H, m); 8.90 (1H, d, J=2.2 Hz); 10.5 (1H, 2s) Elemental analysis: C₁₉H₁₆N₄O₃ (348.36)

| | C % | H % | N % |
|---|---|---|---|
| Calculated | 65.17 | 4.66 | 16.00 |
| Found | 65.28 | 4.62 | 15.84 |

### EXAMPLE 2

### N-(4-Methoxyphenyl)-4-[1-(4-methoxyphenyl)-2-oxohydrazino]benzamide

### a) Ethyl 4-[(4-methoxyphenyl)amino]benzoate

A mixture of 0.545 g (3.3 mmol) of ethyl 4-aminobenzoate, 0.597 g (3.3 mmol) of copper acetate, 1 g (6.6 mmol) of 4-methoxyphenylboronic acid and 0.670 g (6.6 mmol) of triethylamine in 20 ml of dichloromethane is stirred for 24 hours at room temperature. A further 1 g (6.6 mmol) of 4-methoxyphenylboronic acid, 1.19 g (6.6 mmol) of copper acetate and 0.67 g (6.6 mmol) of triethylamine are then added to the medium. After stirring for 48 hours at room temperature, the reaction medium is poured into water and extracted with CH₂Cl₂. After filtering off an insoluble material and separation of the phases by settling, the organic phase is washed with water, dried over Na₂SO₄ and then concentrated under vacuum. The residue, purified by chromatography on a column of silica in a heptane/ethyl acetate mixture (6:1), gives 0.543 g of beige-coloured crystals.

(Yield: 60.7%).

NMR (DMSO-d₆): 1.1 (3H, t, J=7.1 Hz); 3.6 (3H, s); 4.1 (2H, q, J=7.1 Hz); 6.7-6.9 (4H, m); 7.0 (2H, m); 7.6 (2H, d, J=8.8 Hz); 8.4 (1H, s)

IR (KBr): ν=3344 (NH); 1697 (CO)

### b) Ethyl 4-[(t-butoxycarbonyl)(4-methoxyphenyl)amino]benzoate

0.354 g (8.84 mmol) of NaH at 60% in oil is added portionwise, at 10° C., to a solution consisting of 2 g (7.37 mmol) of the compound prepared in Example 2a, in 20 ml of DMF.

After stirring for half an hour at room temperature, a solution of 1.6 g (7.37 mmol) of di-tert-butyl dicarbonate in 10 ml of DMF is added dropwise. The reaction medium is stirred at room temperature for 40 hours and then poured into 300 ml of water, acidified to pH 3 with acetic acid and extracted with ethyl acetate. The organic phase, washed with water and dried over Na₂SO₄, is concentrated under vacuum.

The residue, purified by chromatography on a column of silica in a heptane/ethyl acetate mixture (4:1), gives 2.14 g of a pale yellow oil.

(Yield: 78.4%).

NMR (CDCl₃): 1.35 (3H, t, J=7.1 Hz); 1.4 (9H, s); 3.8 (3H, s); 4.35 (2H, q, J=7.1 Hz); 6.85 (2H, d, J=9.1 Hz); 7.1 (2H, d, J=9.1 Hz); 7.25 (2H, d, J=8.7 Hz); 7.9 (2H, d, J=8.7 Hz).

### c) 4-[(t-Butoxycarbonyl)(4-methoxyphenyl)amino]benzoic acid

A mixture composed of 2.14 g (5.8 mmol) of the ester prepared in Example 2b, 0.387 g (6.9 mmol) of KOH, 28 ml of ethanol and 11 ml of water is stirred for 20 hours at room temperature. After concentration of the ethanol and addition of 60 ml of water, the reaction medium is washed with ether (2×60 ml) and acidified with acetic acid. The precipitate formed is filtered off, washed with water and dried under vacuum to give 1.88 g of a white solid.

(Yield: 94.5%).

NMR (DMSO-d₆): 1.3 (9H, s); 3.7 (3H, s); 6.9 (2H, m); 7.05 (2H, m); 7.2 (2H, m); 7.8 (2H, m); 12.8 (1H, s broad).

### d) t-Butyl 4-methoxyphenyl(4-{[(4-methoxyphenyl)amino]carbonyl}-phenyl)carbamate

0.293 g (2.89 mmol) of triethylamine is added to a solution of 0.51 g (1.48 mmol) of the acid prepared in Example 2c and 0.37 g (1.48 mmol) of bis(2-oxo-3-oxazolidinyl)phosphonyl chloride in 20 ml of diglyme. After stirring for 1.5 hours at 45° C., 0.178 g (1.45 mmol) of 4-methoxyaniline in 2 ml of diglyme is added. The reaction medium is stirred for six hours at 120° C. and then poured into 300 ml of water and extracted with ether (3×200 ml). The organic phase is washed with water, dried over Na₂SO₄ and concentrated under vacuum. After purification by chromatography on a column of silica in a heptane/ethyl acetate mixture (1:1), 0.3 g of a beige-coloured solid is obtained.

(Yield: 45.3%).

NMR (DMSO-d₆): 1.4 (9H, s); 3.75 (3H, s); 3.8 (3H, s); 6.95 (4H, m); 7.15 (2H, m); 7.3 (2H, d, J=8.6 Hz); 7.65 (2H, m); 7.9 (2H, d, J=8.6 Hz); 10.1 (1H, s).

### e) N-(4-Methoxyphenyl)-4-[(4-methoxyphenyl)amino]benzamide

1.25 ml of trifluoroacetic acid are added to a solution of 0.27 g (0.6 mmol) of the compound prepared in Example 2d, in 2.9 ml of CH₂Cl₂. The reaction medium is stirred for 3 hours at room temperature and then poured into water, basified to pH 9 with 1N sodium hydroxide solution and extracted with dichloromethane.

The organic phase, washed with water and dried over Na₂SO₄, is concentrated under vacuum to give a beige-coloured solid.

(Yield: quantitative).

m.p.=145° C.

NMR (DMSO-d₆): 3.75 (6H, 2s); 6.92 (6H, m); 7.1 (2H, d, J=9.0 Hz); 7.65 (2H, d, J=9.1 Hz); 7.8 (2H, d, J=8.7 Hz); 8.35 (1H, s); 9.75 (1H, s).

### f) N-(4-Methoxyphenyl)-4-[1-(4-methoxyphenyl)-2-oxohydrazino]benzamide

Obtained by working as in Example 1b, starting with the compound prepared in Example 2e, to give a pink-beige solid.

(Yield: 89.8%).

m.p.=206-208° C.

NMR (DMSO-d₆) of the 2 conformers: 3.75 (3H, s); 3.8 (3H, 2s); 6.9-7.55 (8H, m); 7.65 (2H, d, J=9 Hz); 8.05 (2H, d, J=8.7 Hz); 10.25 (1H, 2s).

### EXAMPLE 3

### 4-[1-(4-Methoxyphenyl)-2-oxohydrazino]-N-pyrid-3-ylbenzamide

### a) tert-Butyl 4-methoxyphenyl{4-[(pyrid-3-ylamino)carbonyl]phenyl}-carbamate

116 mg (0.9 mmol) of oxalyl chloride are added at room temperature to a solution of 206 mg (0.6 mmol) of 4-[(tert-butoxycarbonyl)(4-methoxyphenyl)-amino]benzoic acid prepared in Example 2c and 5 drops of DMF in 10 ml of CH₂Cl₂. After stirring for 1 hour at room temperature, a further 116 mg (0.9 mmol) of oxalyl chloride are added and the mixture is stirred for 2 hours at room temperature. The reaction medium is then concentrated under vacuum. The residue obtained is taken up in 10 ml of CH₂Cl₂ to which is added a solution composed of 68 mg (0.72 mmol) of 3-aminopyridine and 0.124 g (1.24 mmol) of triethylamine in 10 ml of CH₂Cl₂. After stirring for three days at room temperature, the reaction medium is poured into water and extracted with CH₂Cl₂. The organic phase, washed with water and dried over Na₂SO₄, is concentrated under vacuum. The residue is purified by chromatography on a column of silica in ethyl acetate, to give 96 mg of a beige-coloured solid.

(Yield: 38,1%).

### b) 4-[(4-Methoxyphenyl)amino]-N-pyrid-3-ylbenzamide

Obtained by working as in Example 2c, starting with the compound prepared in Example 3a.

### c) 4-[1-(4-Methoxyphenyl)-2-oxohydrazino]-N-pyrid-3-ylbenzamide

Obtained by working as in Example 1b.

### EXAMPLE 4

### 3-[1-(4-Methoxyphenyl)-2-oxohydrazino]-N-pyrid-3-ylbenzamide

### a) Methyl 3-{[(trifluoromethyl)sulfonyl]oxy}benzoate

4.6 ml (27.5 mmol) of triflic anhydride are added dropwise to a solution of 3.8 9 (25 mmol) of methyl 3-hydroxybenzoate and 5.64 g (27.5 mmol) of 2,6-di-tert-butyl-4-methylpyridine in 91 ml of 1,2-dichloroethane. After stirring for 16 hours at room temperature, the reaction medium is concentrated under vacuum. The residue is taken up in 100 ml of ether. The solvent is filtered off and then concentrated to give an oil, which is purified by chromatography on a column of silica with a CH₂Cl₂/heptane mixture (2:1). 5.9 g of a brown oil are obtained.

(Yield: 83.1%).

NMR (CDCl₃): 3.95 (3H, s); 7.40-7.60 (2H, m); 7.95 (1H, m); 8.05 (1H, m).

### b) Methyl 3-[(4methoxyphenyl)amino]benzoate

A mixture of 5.8 g (20.4 mmol) of the compound prepared in Example 4a, 3.01 g (24.5 mmol) of 4-methoxyaniline, 0.229 g (1.02 mmol) of palladium diacetate, 0.95 g (1.53 mmol) of racemic BINAP and 9.31 g (28.56 mmol) of caesium carbonate in 41 ml of toluene is heated for 10 hours at 80° C., and then poured into 250 ml of water and extracted with ether. The organic phase is washed with water, dried over Na₂SO₄ and then concentrated and purified by chromatography on a column of silica in CH₂Cl₂. 1.79 g of a yellow solid are obtained.

(Yield: 34.1%).

m.p.=120° C.

NMR (CDCl₃): 3.8 (3H, s); 3.9 (3H, s); 5.6 (1H, s broad); 6.9 (2H, m); 7.1 (3H, m); 7.25 (1H, m); 7.45 (1H, m); 7.55 (1H, s)

### c) Methyl 3-[(t-butoxycarbonyl)(4-methoxyphenyl)amino]benzoate

Obtained by working as in Example 2b, starting with the compound prepared in Example 4b. Yellow oil.

(Yield: 26.2%).

NMR (CDCl₃): 1.45 (9H, s); 3.8 (3H, s); 3.9 (3H, s); 6.8-6.9 (2H, m); 7.05-7.15 (2H, m); 7.3-7.45 (2H, m); 7.75-7.85 (1H, m); 7.9 (1H, m)

### d) 3-[(t-Butoxycarbonyl)(4-methoxyphenyl)amino]benzoic acid

Obtained by working as in Example 2c, starting with the compound prepared in Example 4c.

(Yield: 63.6%).

m.p.=162-164° C.

NMR (DMSO-d₆): 1.4 (9H, s); 3.75 (3H, s); 6.85-6.95 (2H, m); 7.15-7.20 (2H, m); 7.45 (2H, m); 7.70 (2H, m); 13.1 (1H, s broad)

### e) t-Butyl 4-methoxyphenyl{3-[(pyrid-3-ylamino)carbonyl]phenyl}-carbamate

Obtained by working as in Example 2d, starting with the compound prepared in Example 4d and 3-aminopyridine. Yellow oil.

(Yield: 49.2%).

NMR (DMSO-d₆): 1.4 (9H, s); 3.75 (3H, s); 6.8-7.6 (6H, m); 7.7 (1H, m); 8.1 (1H, m); 8.3 (1H, m); 8.9 (1H, s); 10.45 (1H, s).

### f) 3-[(4-Methoxyphenyl)amino]-N-pyrid-3-ylbenzamide

Obtained by working as in Example 2e, starting with the compound prepared in Example 4e. Beige-coloured solid.

(Yield: 93.3%).

m.p.=190-192° C.

NMR (DMSO-d₆): 3.8 (3H, s); 6.9 (2H, d, J=8.9 Hz); 7.1 (3H, m); 7.25-7.5 (4H, m); 8.1 (1H, s); 8.15-8.20 (1H, m); 8.3 (1H, m); 8.9 (1H, m); 10.35 (1H, s).

### g) 3-[1-(4-Methoxyphenyl)-2-oxohydrazino]-N-pyrid-3-ylbenzamide

Obtained by working as in Example 1b, starting with the compound prepared in Example 4f. Ochre-coloured solid.

(Yield: 93.0%).

m.p.=60-70° C.

NMR (DMSO-d₆) of the 2 conformers: 3.8 (3H, 2s); 7.05-7.8 (7H, m); 7.95-8.25 (3H, m); 8.35 (1H, m); 8.95 (1H, m); 10.6 (1H, 2s).

### EXAMPLE 5

### 4-[1-(4-Nitrophenyl)-2-oxohydrazino]-N-pyrid-3-ylbenzamide

### a) 4-[(4-Nitrophenyl)amino]-N-pyrid-3-ylbenzamide

A mixture composed of 0.4 g (1.55 mmol) of 4-[(4-nitro-phenyl)amino]benzoic acid (Bach F. L. et al., J. Med. Chem. (1967), 10, 802-806), 0.395 g (1.55 mmol) of bis(2-oxo-3-oxazolidinyl)phosphonyl chloride and 0.314 g (3.1 mmol) of triethylamine in 20 ml of diglyme is heated for half an hour at 40° C., followed by addition of 0.29 9 (3.1 mmol) of 3-aminopyridine in 6 ml of diglyme. The mixture is heated for 6 hours at 120° C. with stirring, adding after the second hour and the fourth hour 0.2 g (0.775 mmol) of bis(2-oxo-3-oxazolidinyl)phosphonyl chloride. The reaction medium is then poured into water and extracted with an ether/ethyl acetate mixture. The organic phase is washed with water and then with saturated NaHCO₃ solution and with water, and then dried over Na₂SO₄ and concentrated to dryness. After purification on a column of silica in a CH₂Cl₂/EtOAc mixture (1:1), 0.134 g of an orange-coloured solid is obtained.

(Yield: 25.8%).

IR (KBr): ν=3366 (NH); 1695 (CO).

NMR (DMSO-d₆): 7.3 (2H, d, J=9.2 Hz); 7.4 (3H, m); 8.05 (2H, d, J=8.6 Hz); 8.2 (3H, m); 8.35 (1H, d, J=3.7 Hz); 9.0 (1H, s); 9.7 (1H, s, exchangeable with D₂O); 10.4 (1H, s, exchangeable with D₂O).

### b) 4-[1-(4-Nitrophenyl)-2-oxohydrazino]-N-pyrid-3-ylbenzamide

Obtained by working as in Example 1b, starting with the compound prepared in Example 5a. Yellow solid.

(Yield: 39.4%).

IR (KBr): ν=1676 (CO).

NMR (DMSO-d₆): 7.4-7.8 (5H, m); 8.15-8.3 (3H, m); 8.35-8.6 (3H, m); 9.0 (1H, s); 10.7 (1H, 2 s broad, exchangeable with D₂O).

### EXAMPLE 6

### 4-[1-(4-Methoxyphenyl)-2-oxohydrazino]-N-[2-(4-methylpiperazin-1-yl)-ethyl]benzamide

### a) 4-(4-Methoxyphenylamino)benzonitrile

3 g (16.5 mmol) of 4-bromobenzonitrile, 2.43 ml (0.24 mmol) of tri-tert-butylphosphine dissolved in toluene (1 g/50 ml) and 12.5 ml of toluene are added to a mixture consisting of 1.85 g (15 mmol) of 4-methoxyaniline, 0.172 g (0.3 mmol) of bis(dibenzylideneacetone)palladium and 2.16 g (22.5 mmol) of sodium tert-butoxide. After stirring for 2 hours at room temperature, the reaction medium is poured into ice-cold water and then extracted with ether. The organic phase is washed with water, dried over Na₂SO₄ and then concentrated under vacuum to give 3.5 g of solid residue. After purification by chromatography on a column of silica in a hexane/dichloromethane mixture (2:3), 3 g of a pale yellow solid are obtained.

(Yield: 89.3%).

IR (KBr): ν=3386 (NH); 2228 (CN).

NMR (CDCl₃): 3.95 (3H, s); 6.0 (1H, s, exchangeable with D₂O); 6.9 (2H, m); 7.05 (2H, m); 7.25 (2H, m); 7.55 (2H, m).

This compound was also obtained by reacting 1.21 g (10 mmol) of 4-fluorobenzonitrile, 1.23 g (10 mmol) of 4-methoxyaniline and 1.7 g (15 mmol) of potassium tert-butoxide in 10 ml of DMSO, for 24 hours at room temperature. After work-up, 0.88 g of the expected compound is obtained (yield: 39%).

### b) 4-(4-Methoxyphenylamino)benzoic acid

A mixture of 3 g (13.4 mmol) of the compound prepared in Example 6a, 1.5 g (26.8 mmol) of KOH and 80 ml of ethylene glycol is refluxed for 4 hours. After cooling, the reaction medium is poured into ice-cold water and acidified with acetic acid. The precipitate formed is suction-filtered, rinsed with water and dried at 50° C. to give 2.9 g of a beige-coloured solid.

(Yield: 89.2%).

m.p.=170° C.

IR (KBr): ν=3403 (NH); 1675 (CO).

NMR (DMSO-d₆): 3.7 (3H, s); 6.8-7.0 (4H, m); 7.1 (2H, d, J=8.8 Hz); 7.8 (2H, d, J=8.8 Hz); 8.5 (1H, s exchangeable with CF₃COOD); 12.2 (1H, s broad, exchangeable with CF₃COOD).

### c) 4-[(4-Methoxyphenyl)amino]-N-[2-(4-methylpiperazin-1-yl)ethyl]benzamide

97.3 mg (0.4 mmol) of the compound prepared in Example 6b are added to a suspension of 400 mg of commercial triphenylphosphine on polymer (3 mmol/g) in 1.1 ml of dichloromethane, followed by addition of 0.048 ml (0.48 mmol) of trichloroacetonitrile. After stirring for 3 hours at room temperature, the reaction medium is filtered and the filtrate is poured into a suspension of 329.7 mg of commercial N-methylmorpholine on polymer (3.64 mmol/g) and 62.9 mg (0.4 mmol) of 2-(4-methylpiperazin-1-yl)ethylamine in 2.2 ml of THF. The new suspension is stirred for 16 hours at room temperature and then filtered. The filtrate is concentrated under vacuum to give 110 mg of solid.

(Yield: 74.6%).

NMR (CDCl₃): 1.8 (2H, m); 2.25 (3H, s); 2.4-2.8 (8H, m); 3.5 (2H, m); 3.8 (3H, s); 5.8 (1H, s); 6.8 (4H, m); 7.05 (2H, m); 7.5-7.7 (3H, m).

### d) 4-[1-(4-Methoxyphenyl)-2-oxohydrazino]-N-[2-(4-methylpiperazin-1-yl)-ethyl]benzamide

Obtained by working as in Example 1b, starting with the compound prepared in Example 6c.

(Yield: 34.8%).

NMR (CDCl₃): 1.6-1.8 (2H, m); 2.1 (3H, s); 2.3-2.7 (8H, m); 3.5 (2H, m); 3.8 (3H, 2s); 6.8-7.0 (4H, m); 7.4 (2H, d, J=8.7 Hz); 7.9 (2H, d, J=8.7 Hz); 8.3 (1H, s broad).

Tables D to F below illustrate the preparation of the compounds 7a to 139a of the formula II and the preparation of the compounds 7b to 139b of the formula I.

TABLE D

| | Formula II |
|---|---|
| 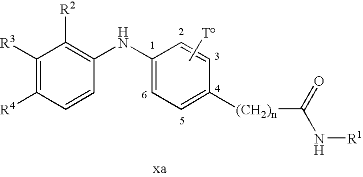 | |
| | Formula I |
| 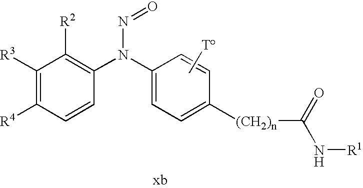 | |

| Examples x | R¹ | R² | R³ | R⁴ | T° | n |
|---|---|---|---|---|---|---|
| 7 | 3-trifluoromethylphenyl | H | H | —OCH₃ | H | 0 |
| 8 | 3-nitrophenyl | H | H | —OCH₃ | H | 0 |
| 9 | 3-pyridylmethyl | H | H | —OCF₃ | H | 0 |
| 10 | 3-pyridylmethyl | H | H | —S—CH₃ | H | 0 |
| 11 | phenyl | H | H | —CH₃ | H | 0 |
| 12 | phenylmethyl | H | H | —CH₃ | H | 0 |
| 13 | 3-pyridyl | H | H | —SO₂—CH₃ | H | 0 |
| 14 | 2-pyridyl | H | H | —OCH₃ | H | 0 |
| 15 | 4-pyridyl | H | H | —OCH₃ | H | 0 |
| 16 | N,N-diisopropylaminoethyl | H | H | —SO₂CH₃ | H | 0 |
| 17 | 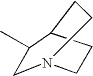 | H | H | —SO₂CH₃ | H | 0 |
| 18 | 1-imidazolyl-n-propyl | H | H | —SO₂CH₃ | H | 0 |
| 19 | N-methyl-2-pyrrolidinyl-ethyl | H | H | —SO₂CH₃ | H | 0 |
| 20 | N-methyl-4-piperazinyl-n-propyl | H | H | —SO₂CH₃ | H | 0 |
| 21 | Phenyl-1-allyle | H | H | —OCH₃ | H | 0 |
| 22 | 3-pyridylmethyl | H | H | —SO₂CH₃ | H | 0 |
| 23 | N,N-dimethylamino-n-propyl | H | H | —SO₂CH₃ | H | 0 |
| 24 | 4-morpholinophenyl | —OCH₃ | H | H | H | 0 |
| 25 | 4-morpholinophenyl | H | Cl | H | H | 0 |
| 26 | 4-morpholinophenyl | H | H | —SCH₃ | H | 0 |
| 27 | 4-morpholinophenyl | H | H | —CF₃ | H | 0 |
| 28 | 3-pyridyl | H | H | —CF₃ | H | 0 |
| 29 | 3-pyridylmethyl | H | Cl | H | H | 0 |
| 30 | 3-pyridylmethyl | H | H | —OCH₃ | H | 0 |
| 31 | 3-pyridylmethyl | H | H | —CH₃ | H | 0 |
| 32 | 3-pyridylmethyl | H | H | —F | H | 0 |
| 33 | phenyl | H | H | —OCH₃ | H | 0 |
| 34 | phenyl | H | H | —SCH₃ | H | 0 |
| 35 | phenyl | H | H | —CF₃ | H | 0 |
| 36 | 4-N,N-dimethylaminophenyl | —OCH₃ | H | H | H | 0 |
| 37 | 4-N,N-dimethylaminophenyl | H | H | F | H | 0 |
| 38 | 4-methylthiophenyl | H | Cl | H | H | 0 |
| 39 | 4-methylthiophenyl | H | H | —OCF₃ | H | 0 |
| 40 | 4-methylthiophenyl | H | H | —F | H | 0 |
| 41 | phenylmethyl | —OCH₃ | H | H | H | 0 |
| 42 | phenylmethyl | H | —Cl | H | H | 0 |
| 43 | phenylmethyl | H | H | —SCH₃ | H | 0 |
| 44 | phenylmethyl | H | H | —OCF₃ | H | 0 |
| 45 | phenylmethyl | H | H | —F | H | 0 |
| 46 | 4-trifluoromethoxyphenyl | H | H | —NMe₂ | H | 0 |
| 47 | phenyl | —H | —Cl | —H | H | 0 |
| 48 | phenyl | H | H | —OCF₃ | H | 0 |
| 49 | 4-cyanophenyl | H | H | —CF₃ | H | 0 |
| 50 | 4-fluorophenyl | —OCH₃ | H | H | H | 0 |
| 51 | 4-fluorophenyl | H | —Cl | H | H | 0 |
| 52 | 4-N,N-dimethylaminophenyl | H | H | —CH₃ | H | 0 |
| 53 | 4-trifluoromethoxyphenyl | H | H | F | H | 0 |
| 54 | N,N-dimethylamino-n-propyl | H | H | —OCH₃ | H | 0 |
| 55 | 1-imidazolyl-n-propyl | H | H | —OCH₃ | H | 0 |
| 56 | N-methylpyrrolidinyl | H | H | —OCH₃ | H | 0 |
| 57 | N,N-diisopropylthyl | H | H | —OCH₃ | H | 0 |
| 58 | 5-pyrimidinyl | H | H | —OCH₃ | H | 0 |
| 59 | 4-morpholinophenyl | H | H | —CH₃ | H | 0 |
| 60 | 4-morpholinophenyl | H | H | —F | H | 0 |
| 61 | 3-pyridylmethyl | —OCH₃ | H | H | H | 0 |
| 62 | 4-N,N-dimethylaminophenyl | H | H | —SCH₃ | H | 0 |
| 63 | phenylmethyl | H | H | —OCH₃ | H | 0 |
| 64 | phenylmethyl | H | H | —N(CH₃)₂ | H | 0 |
| 65 | 4-trifluoromethylphenylmethyl | H | Cl | H | H | 0 |
| 66 | 4-morpholinophenyl | —OCH₃ | H | H | 3-F | 0 |
| 67 | 4-morpholinophenyl | H | Cl | H | 3-F | 0 |
| 68 | 4-morpholinophenyl | H | H | —OCH₃ | 3-F | 0 |
| 69 | 4-morpholinophenyl | H | H | —OCF₃ | 3-F | 0 |
| 70 | 4-morpholinophenyl | H | H | —CH₃ | 3-F | 0 |
| 71 | 4-morpholinophenyl | H | H | F | 3-F | 0 |
| 72 | 3-pyridyl | —OCH₃ | H | H | 3-F | 0 |
| 73 | 3-pyrldyl | H | Cl | H | 3-F | 0 |
| 74 | 3-pyridylmethyl | H | Cl | H | 3-F | 0 |
| 75 | 3-pyridylmethyl | H | H | —OCH₃ | 3-F | 0 |
| 76 | 3-pyridylmethyl | H | H | —CF₃ | 3-F | 0 |
| 77 | 3-pyridylmethyl | H | H | —CH₃ | 3-F | 0 |
| 78 | phenyl | H | H | —OCF₃ | 3-F | 0 |
| 79 | phenyl | H | H | —CF₃ | 3-F | 0 |
| 80 | phenyl | H | H | —CH₃ | 3-F | 0 |
| 81 | 4-N,N-dimethylaminophenyl | —OCH₃ | H | H | 3-F | 0 |
| 82 | 4-N,N-dimethylaminophenyl | H | —Cl | H | 3-F | 0 |
| 83 | 4-N,N-dimethylaminophenyl | H | H | —SCH₃ | 3-F | 0 |
| 84 | 4-N,N-dimethylaminophenyl | H | H | —OCF₃ | 3-F | 0 |
| 85 | 4-N,N-dimethylaminophenyl | H | H | —CH₃ | 3-F | 0 |
| 86 | 4-N,N-dimethylaminophenyl | H | H | —F | 3-F | 0 |
| 87 | 4-methylthiophenyl | H | H | —F | 3-F | 0 |
| 88 | 4-trifluoromethoxyphenylmethyl | H | H | —CH₃ | 3-F | 0 |
| 89 | 4-trifluoromethoxyphenylmethyl | H | H | —F | 3-F | 0 |
| 90 | 4-morpholinophenyl | H | H | —CF₃ | 3-F | 0 |
| 91 | 3-pyridyl | H | H | —OCF₃ | 3-F | 0 |
| 92 | 4-cyanophenyl | H | H | —CF₃ | 3-F | 0 |
| 93 | 4-fluorophenyl | H | H | —OCF₃ | 3-F | 0 |
| 94 | 3-pyridylmethyl | H | H | —SCH₃ | 3-F | 0 |
| 95 | 3-pyridylmethyl | H | H | —OCF₃ | 3-F | 0 |
| 96 | 3-pyridylmethyl | H | H | —F | 3-F | 0 |
| 97 | 4-methylthiophenyl | H | H | —CF₃ | 3-F | 0 |
| 98 | 4-trifluoromethoxyphenyl | H | H | —CF₃ | 3-F | 0 |
| 99 | 4-morpholinophenyl | —OCH₃ | H | H | H | 1 |
| 100 | 4-morpholinophenyl | H | Cl | H | H | 1 |
| 101 | 4-morpholinophenyl | H | H | —OCH₃ | H | 1 |
| 102 | 4-morpholinophenyl | H | H | —SCH₃ | H | 1 |
| 103 | 4-morpholinophenyl | H | H | —CH₃ | H | 1 |
| 104 | 4-morpholinophenyl | H | H | —F | H | 1 |
| 105 | 4-fluorophenyl | H | H | —OCH₃ | H | I |
| 106 | 4-fluorophenyl | H | H | —F | H | 1 |
| 107 | 4-N,N-dimethylaminophenyl | H | H | —OCF₃ | H | 1 |
| 108 | 4-N,N-dimethylaminophenyl | H | H | —F | H | 1 |
| 109 | 4-methylthiophenyl | —OCH₃ | H | H | H | 1 |
| 110 | 4-methylthiophenyl | H | Cl | H | H | 1 |
| 111 | 4-methylthiophenyl | H | H | —CH₃ | H | 1 |
| 112 | phenylmethyl | H | Cl | H | H | 1 |
| 113 | phenylmethyl | H | H | —OCH₃ | H | 1 |
| 114 | phenylmethyl | H | H | —SCH₃ | H | 1 |
| 115 | phenylmethyl | H | H | —CH₃ | H | 1 |
| 116 | 4-trifluoromethoxyphenylmethyl | —OCH₃ | H | H | H | 1 |
| 117 | 4-trifluoromethoxyphenylmethyl | H | Cl | H | H | 1 |
| 118 | 4-trifluoromethoxyphenylmethyl | H | H | —SCH₃ | H | 1 |
| 119 | 4-trifluoromethoxyphenylmethyl | H | H | —OCF₃ | H | 1 |
| 120 | 4-trifluoromethoxyphenylmethyl | H | H | F | H | 1 |
| 121 | phenyl | H | H | F | H | 1 |
| 122 | 4-fluorophenyl | —OCH₃ | H | H | H | 1 |
| 123 | 4-fluorophenyl | H | Cl | H | H | 1 |
| 124 | 4-fluorophenyl | H | H | —OCF₃ | H | 1 |
| 125 | 4-fluorophenyl | H | H | —CH₃ | H | 1 |
| 126 | 4-N,N-dimethylaminophenyl | H | H | —SCH₃ | H | 1 |
| 127 | 4-methylthiophenyl | H | H | —SCH₃ | H | 1 |
| 128 | phenylmethyl | —OCH₃ | H | H | H | 1 |
| 129 | phenylmethyl | —H | H | —OCF₃ | H | 1 |
| 130 | 4-trifluoromethoxyphenylmethyl | H | H | —CH₃ | H | 1 |

TABLE E

| | |
|---|---|
| | Formula III |
| 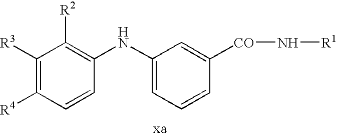 | |
| | Formula III |
| 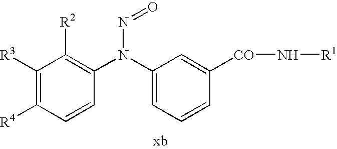 | |

| Examples | R¹ | R² | R³ | R⁴ |
|---|---|---|---|---|
| 131 | phenylmethyl | H | Cl | H |
| 132 | 3-pyridylmethyl | H | H | —OCH₃ |
| 133 | 5-pyridiminyl | H | H | —OCH₃ |
| 134 | 2-pyridyl | H | H | —OCH₃ |
| 135 | 4-pyridyl | H | H | —OCH₃ |

The spectral characterisation data of some of the compounds of the invention are detailed below:

### EXAMPLE 7a

(DMSO-d6): 3.75 (3H, s); 6.95 (4H, d, J=8.5 Hz); 7.15 (2H, m); 7.4 (1H, d, J7.7 Hz); 7.55 (1H, m); 7.8 (2H, m); 8.0 (1H, d, J=8.1 Hz); 8.25 (1H, s); 8.45 (1H, s); 10.2 (1H, s).

### EXAMPLE 8a

(DMSO-d6): 3.75 (3H, s); 6.95 (4H, 2d, J=2.3 Hz and 9.1 Hz); 7.15 (2H, d, J=8.7 Hz); 7.65 (1H, t, J=8.3 Hz); 7.85 (2H, d, J=9 Hz); 7.9 (1H, m); 8.2 (1H, m); 8.45 (1H, s);8.8 (1H, s); 10.35 (1H, s).

### EXAMPLE 7b

2 conformers (DMSO-d6)=3.8 (3H, 2s); 7.05-7.7 (8H, m); 8.0-8.35 (4H, m); 10.65 (1H, 2s).

### EXAMPLE 8b

2 conformers (DMSO-d6)=3.85 (3H, 2s); 6.8-8.25 (11H, m); 8.8 (1H, m); 10.8 (1H, 2s).

TABLE F

| | |
|---|---|
| | Formula II |
| 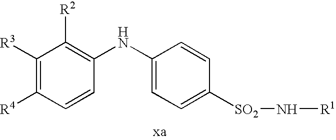 | |
| | Formula I |
| 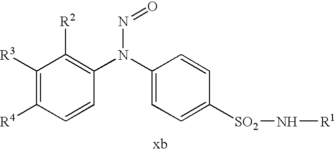 | |

| Example x | R¹ | R² | R³ | R⁴ |
|---|---|---|---|---|
| 136 | 3-pyridyl | H | H | —OCH₃ |
| 137 | 3-pyridyl | H | H | —CN |
| 138 | 3-pyridylmethyl | H | H | —OCH₃ |

### EXAMPLE 139

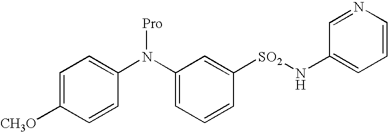

### EXAMPLE 139a

### Pro=H

### EXAMPLE 139b

### Pro=N═O

### EXAMPLE 140

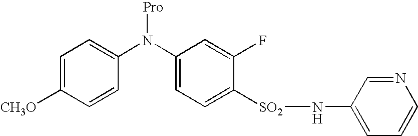

### EXAMPLE 140a

Pro=H

(DMSO-d6)=3.73 (3H, s); 6.55 (1H, dd, J=2.3 and 13.6 Hz); 6.60 (1H, dd, J=2.2 and 8.7 Hz); 6.93 (2H, m); 7.10 (2H, m); 7.27 (1H, m); 7.44 (1H, m); 7.55 ((1H, t, J=8.7 Hz); 8.20 (1H, m); 8.30 (1H, d, J=2.3 Hz); 8.81 (1H, s broad); 10.52 (1H, s broad).

### EXAMPLE 140b

Pro=NO

(DMSO-d6)=3.80 (3H, 2s); 7.0-7.6 (8H, m); 7.85-8.05 (1H, m); 8.20-8.40 (2H, m); 10.98 (1H, s broad).

### EXAMPLE 141

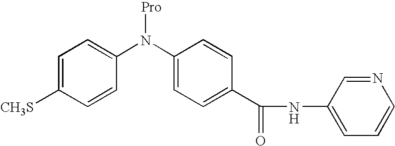

### EXAMPLE 141a

Pro=H

(DMSO-d6)=2.44 (3H, s); 7.08 (2H, d, J=8.7 Hz); 7.15 (2H, d, J=8.6 Hz); 7.26 (2H, d, J=8.6 Hz); 7.35 (1H, m); 7.88 (2H, d, J=8.7 Hz); 8.17 (1H, m); 8.26 (1H, m); 8.68 (1H, s); 8.90 (1H, d, J=1.8 Hz); 10.14 (1H, s).

### EXAMPLE 141b

Pro=NO

(DMSO-d6)=2.45 (3H, s); 7.05-7.20 (2H, m); 7.30-8.10 (10H, m); 10.35 (1H, 2s broadened).

### EXAMPLE 142

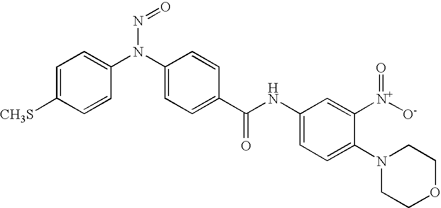

LC-MS (ES+): 494.2 (M+H)

(ES−): 492.2 (M−H)

(DMSO-d6)=2.53 (3H, s); 2.95 (4H, m); 7.10-7.60 (7H, m); 7.90-8.15 (3H, m); 8.35 (1H, m); 10.59 (1H, s broad).

TABLE G

| Example | | |
|---|---|---|
| No. | ¹H-NMR | LC-MS |
| 7a | (DMSO-d6): 3.75(3H, s); 6.95(4H, d, | — |
| | J=8.5Hz); 7.15(2H, m); 7.4(1H, d, J=7.7Hz); | |
| | 7.55(1H, m); 7.8(2H, m); 8.0 | |
| | (1H, d, J=8.1Hz); 8.25(1H, s); 8.45 | |
| | (1H, s); 10.2(1H, s) | |
| 8a | (DMSO-d6): 3.75(3H, s); 6.95(4H, | — |
| | 2d, J=2.3Hz and 9.1Hz); 7.15(2H, | |
| | d, J=8.7Hz); 7.65(1H, t, J=8.3Hz); | |
| | 7.85(2H, d, J=9Hz); 7.9(1H, m); 8.2 | |
| | (1H, m); 8.45(1H, s); 8.8(1H, s); | |
| | 10.35(1H, s) | |
| 9a | — | (ES+) = 388.34(M + H) |
| | | (ES−) = 386.36(M − H) |
| 10a | — | (ES+) = 350.33(M + H) |
| 11a | — | (ES+) = 303.30(M + H) |
| 12a | — | (ES+) = 317.32(M + H) |
| 13a | (DMSO-d6) = 3.14(3H, s); 7.12-9.08 | — |
| | (12H, m); 9.29(1H, s); 10.28(1H, s) | |
| 14a | (DMSO-d6) = 3.74(3H, s); 6.75-8.56 | — |
| | (13H, m); 10.31(1H, s broadened) | |
| 15a | (DMSO-d6) = 3.74(3H, s); 6.81-8.61 | — |
| | (12H, m+1H, s); 10.21(1H, s) | |
| 16a | (DMSO-d6) = 0.96(12H, m); 1.1-1.85 | — |
| | (2H, m); 2.95(2H, m); 3.12(3H, s); | |
| | 3.18(2H, m); 7.06-7.34(4H, m); 7.59-7.92 | |
| | (4H, m); 8.20(1H, m broadened); | |
| | 9.14(1H, s) | |
| 17a | (DMSO-d6) = 0.83-4.01(12H, m); 3.12 | — |
| | (3H, s); 7.05-7.99(8H, m); 8.09(1H, m | |
| | broadened); 9.18(1H, s) | |
| 18a | (DMSO-d6) = 1.94(2H, m); 2.97-3.27 | — |
| | (3H, s+2H, m); 4.01(2H, m); 6.73-7.97 | |
| | (11H, m); 8.36(1H, m); 9.17(1H, s | |
| | broadened) | |
| 19a | (DMSO-d6) = 0.87-2.10(8H, m); 2.19 | — |
| | (3H, s); 2.92(1H, m); 3.12(3H, s); | |
| | 3.27(2H, m); 6.97-7.95(8H, m); 8.33 | |
| | (1H, m); 9.19(1H, s broadened) | |
| 20a | (DMSO-d6) = 1.67(4H, m); 2.13(3H, | — |
| | s); 2.31(8H, m); 3.12(3H, s); 3.26 | |
| | (2H, m); 7.04-7.94(8H, m); 8.34(1H, | |
| | m); 9.16(1H, s) | |
| 21a | (DMSO-d6) = 3.72(3H, s); 4.02(2H, | — |
| | m); 6.22-7.79(15H, m); 8.25(1H, s); | |
| | 8.38(1H, m) | |
| 22a | (DMSO-d6) = 3.12(3H, s); 4.47(2H, | — |
| | m); 7.07-8.69(12H, m); 8.94(1H, m); | |
| | 9.18(1H, s) | |
| 23a | (DMSO-d6) = 1.62(2H, m); 2.11(6H, | |
| | s); 2.23(2H, m); 3.11(3H, s); 3.25 | |
| | (2H, m); 6.95-7.96(8H, m); 8.35(1H, | |
| | m); 9.18(1H, s broadened) | |
| 24a | — | (ES+) = 404.3(M + H) |
| 25a | — | (ES+) = 408.3/410.3 |
| | | (M + H with a heavy-isotope |
| | | chlorine atom) |
| 26a | — | (ES+) = 420.3(M + H) |
| 27a | — | (ES+) = 442.4(M + H) |
| 28a | — | (ES+) = 358.2(M + H) |
| | | (ES−) = 356.2(M − H) |
| 29a | — | (ES+) = 338.2/340.2(M + H |
| | | with a chlorine atom); |
| | | (ES−) = 336.2/338.2(M − H |
| | | with a chlorine atom) |
| 30a | — | (ES+) = 334.34(M + H) |
| | | (ES−) = 332.35(M − H) |
| 31a | — | (ES+) = 318.3(M + H) |
| | | (ES−) = 316.2(M − H) |
| | | 362.2(M + HCOO-adduct) |
| 32a | — | (ES+) = 322.2(M + H) |
| | | (ES−) = 320.2(M − H) |
| | | 366.2(M + HCOO-adduct) |
| 33a | — | (ES+) = 319.32(M + H) |
| | | (ES−) = 317.34(M − H) |
| 34a | — | (ES+) = 335.32(M + H) |
| | | (ES−) = 333.32(M − H) |
| 35a | — | (ES+) = 357.3(M + H) |
| | | (ES−) = 355.3(M − H) |
| 36a | — | (ES+) = 362.4(M + H) |
| 37a | — | (ES+) = 350.4(M + H) |
| | | (ES−) = 348.3(M − H) |
| | | 394.4(M + HCOO-adduct) |
| 38a | — | (ES+) = 369.2/371.2 |
| | | (M + H with a chlorine atom) |
| | | (ES−) = 367.2/369.3(M − H |
| | | with a chlorine atom) |
| 39a | — | (ES+) = 419.3(M + H) |
| | | (ES−) = 417.3(M − H) |
| | | 463.3(M + HCOO-adduct) |
| 40a | — | (ES+) = 353.3(M + H) |
| | | (ES−) = 351.1(M − H) |
| 41a | — | (ES+) = 333.3(M + H) |
| | | 665.6(dimer+H) |
| 42a | — | (ES+) = 337.26(M + H) |
| 43a | — | (ES+) = 349.3(M + H) |
| | | 697.6(dimer+H) |
| 44a | — | (ES+) = 387.3(M + H) |
| | | 773.6(dimer+H) |
| 45a | — | (ES+) = 321.3(M + H) |
| | | 641.5(dimer+H) |
| | | (ES−) = 319.3(M − H) |
| | | 365.3(M + HCOO-adduct) |
| 46a | — | (ES+) = 430.4(M + H) |
| 47a | — | (ES+) = 323.25(M + H) |
| | | (ES−) = 321.25(M − H) |
| 48a | — | (ES+) = 373.2(M + H) |
| | | (ES−) = 371.2(M − H) |
| | | 417.3(M + HCOO-adduct) |
| 49a | — | (ES+) = 382.3(M + H) |
| | | (ES−) = 380.3(M − H) |
| 50a | — | (ES+) = 337.3(M + H) |
| | | (ES−) = 335.3(M − H) |
| | | 381.3(M + HCOO-adduct) |
| 51a | — | (ES+) = 341.3/343.3 |
| | | (M + H with a chlorine atom) |
| | | (ES−) = 339.2/341.3(M − H |
| | | with a chlorine atom) |
| 52a | — | (ES+) = 346.4(M + H) |
| 53a | — | (ES+) = 405.3(M + H) |
| | | (ES−) = 403.3(M − H) |
| | | 449.4(M + HCOO-adduct) |
| 54a | (DMSO-d6) = 1.7(2H, m); 2.2(6H, 2s); | — |
| | 2.4(2H, m); 3.2(2H, m); 3.8(3H, s); | |
| | 6.9-7.1(4H, m); 7.2-7.3(2H, m); 7.7 | |
| | (2H, m); 8.25(1H, t, J=5.28Hz); 8.4 | |
| | (1H, s) | |
| 55a | (DMSO-d6) = 1.8-2.1(4H, m); 3.8(3H, | — |
| | s); 4.1(2H, m); 6.9-7.1(4H, m); 7.2-7.4 | |
| | (3H, m); 7.6-7.85(4H, m); 8.3(1H, | |
| | t, J=5.5Hz); 8.4(1H, s) | |
| 56a | (DMSO-d6) = 1.4-3.6(14H, m); 3.8 | — |
| | (3H, s); 6.8-7.0(4H, m); 7.1-7.2(2H, | |
| | m); 7.6-7.7(2H, m); 8.2(1H, t, J=5.48Hz); | |
| | 8.3(1H, s) | |
| 57a | (DMSO-d6) = 1.1(12H, m); 2.7(4H, | — |
| | m); 3.1(2H, m); 3.5(3H, s); 3.9(1H, | |
| | m); 6.9-7.1(4H, m); 7.3(2H, m); 7.8 | |
| | (2H, m); 8.4(1H, s) | |
| 58a | (DMSO-d6) = 3.74(3H, s); 6.93(4H, | — |
| | m); 7.13(2H, m); 7.85(2H, m); 8.5 | |
| | (1H, m); 8.87(1H, s); 9.15(2H, s); | |
| | 10.33(1H, s broad) | |
| 59a | — | (ES+) = 388.4(M + H) |
| 60a | — | (ES+) = 392.4(M + H) |
| 61a | — | (ES+) = 334.3(M + H) |
| 62a | — | (ES+) = 378.3(M + H) |
| 63a | — | (ES+) = 333.3(M + H) |
| | | dimer 665.6(2M + H) |
| 64a | — | (ES+) = 346.4(M + H) |
| 65a | — | (ES+) = 421.3/423.3(M + H) |
| | | with a chlorine atom |

TABLE H

| Example No. | ¹H-NMR | LC-MS |
|---|---|---|
| 7b | 2 conformers(DMSO-d6)=3.8 | — |
| | (3H, 2s); 7.05-7.7 | |
| | (8H, m); 8.0-8.35(4H, m); | |
| | 10.65(1H, 2s) | |
| 8b | 2 conformers(DMSO-d6)=3.85 | — |
| | (3H, 2s); 6.8-8.25 | |
| | (11H, m); 8.8(1H, m); | |
| | 10.8(1H, 2s) | |
| 9b | (DMSO-d6)=4.55(2H, | — |
| | m); 7.05-7.65(7H, m), | |
| | 7.80-8.10(3H, m); 8.45-8.65 | |
| | (2H, m); 9.25(1H, 2t, | |
| | J=5.75Hz) | |
| 10b | (DMSO-d6)=2.5(3H, s); | — |
| | 4.52(2H, m); 7.11(1H, d, | |
| | J=8.5Hz); 7.20-8.1(9H, | |
| | m); 8.40-8.65(2H, m); | |
| | 9.25(1H, 2t, J=5.6Hz) | |
| 11b | (DSMO-d6)=2.2(3H, s); | — |
| | 7.0-7.6(6H, m); 7.7-7.25 | |
| | (5H, m); 8.0-8.15(2H, m); | |
| | 10.35(1H, 2s broadened) | |
| 12b | (DMSO-d6)=2.35(3H, | — |
| | 2s); 4.5(2H, m); 7.05(1H, | |
| | d, J=8.2Hz); 7.2-7.5(9H, | |
| | m); 7.65-8.1(3H, m); 9.15 | |
| | (1H, 2t, J=6Hz) | |
| 13b | (DMSO-d6)=3.26(3H, s); | — |
| | 7.16-8.48(12H, m); | |
| | 10.43-10.81(1H, 2s | |
| | broadened) | |
| 14b | (DMSO-d6)=3.82(3H, | — |
| | 2s); 6.85-8.55(12H, m); | |
| | 10.72-11.08(1H, 2s | |
| | broadened) | |
| 15b | (DMSO-d6)=3.82(3H, | — |
| | 2s); 6.89-8.63(12H, m); | |
| | 10.52-10.87(1H, 2s | |
| | broadened) | |
| 16b | (DMSO-d6)=0.97(12H, | — |
| | m); 2.97(2H, m); 3.29-4.19 | |
| | (7H, m); 7.05-8.70 | |
| | (8H, m) | |
| 17b | (DMSO-d6)=1.20-1.92 | — |
| | (6H, m); 2.65(4H, m); | |
| | 2.86(1H, m); 3.12(3H, s); | |
| | 3.91(1H, m); 7.10-8.23 | |
| | (8H, m) | |
| 18b | (DMSO-d6)=1.96(2H, | — |
| | m); 3.25(2H, m+3H, s); | |
| | 4.03(2H, m); 6.76-8.27 | |
| | (11H, m); 8.52-8.82(1H, | |
| | split, m broadened) | |
| 19b | (DMSO-d6)=0.96-2.37 | — |
| | (13H, m); 2.93(1H, m); | |
| | 3.25(3H, s); 7.11-8.34 | |
| | (8H, m); 8.52-8.81(1H, | |
| | 2m broadened) | |
| 20b | (DMSO-d6)=0.94-2.45 | — |
| | (17 H, m); 3.25(3H, s); | |
| | 7.14-8.26(8H, m); 8.52-8.76 | |
| | (1H, 2m broadened) | |
| 21b | (DMSO-d6)=3.82(3H, | — |
| | 2s); 4.08(2H, m); 6.18-8.19 | |
| | (15H, m); 8.68-9.09 | |
| | (1H, 2m broadened) | |
| 22b | (DMSO-d6)=3.25(3H, s); | — |
| | 4.51(2H, m), 7.09-8.65 | |
| | (12H, m); 9.06-9.43(1H, | |
| | 2m broadened) | |
| 23b | (DMSO-d6)=0.28-3.89 | — |
| | (15 H, m); 6.56-7.16(8H, | |
| | m) | |
| 24b | (DMSO-d6)=0.64-3.68 | — |
| | (11H, m); 6.75-8.33(12H, | |
| | m); 10.00-10.68(1H, 2s | |
| | broadened) | |
| 25b | (DSMO-d6)=1.12-3.89 | — |
| | (8H, m); 6.75-8.49(12H, | |
| | m); 10.05-10.85(1H, 2s | |
| | broadened) | |
| 26b | (DMSO-d6)=1.11-3.92 | — |
| | (11H, m); 6.71-8.22(12H, | |
| | m); 9.97-10.31(1H, 2s | |
| | broadened) | |
| 27b | (DMSO-d6)=1.05-3.93 | — |
| | (8H, m); 6.87-8.58(12H, | |
| | m); 10.16-10.86(1H, 2s | |
| | broadened) | |
| 28b | (DMSO-d6)=7.02-8.52 | — |
| | (12H, m); 10.43-10.75 | |
| | (1H, 2s broadened) | |
| 29b | (DMSO-d6)=4.51(2H, | — |
| | m); 6.91-8.68(12H, m); | |
| | 9.05-9.38(1H, 2m broadened) | |
| 30b | (DMSO-d6)=3.81(3H, | — |
| | 2s); 4.49(2H, m); 6.83-8.65 | |
| | (12H, m); 9.19(1H, | |
| | 2m broadened) | |
| 31b | (DMSO-d6)=2.12-2.95 | — |
| | (3H, 2s); 4.49(2H, m); | |
| | 6.90-8.69(12H, m); 8.86-9.33 | |
| | (1H, 2m broadened) | |
| 32b | (DMSO-d6)=4.51(2H, | — |
| | m); 6.95-8.73(12H, m); | |
| | 8.99-9.36(1H, 2m broadened) | |
| 33b | (DMSO-d6)=3.82(3H, | — |
| | 2s); 6.66-8.44(13 H, m); | |
| | 10.06-10.47(1H, 2s | |
| | broadened) | |
| 34b | (DMSO-d6)=2.44(3H, | — |
| | 2s); 6.81-8.31(13H, m); | |
| | 10.31(1H, 2s broadened) | |
| 35b | (DMSO-d6)=6.86-8.45 | — |
| | (13H, m); 10.18-10.55 | |
| | (1H, 2s broadened) | |
| 36b | (DMSO-d6)=2.70-2.89 | — |
| | (6H, s); 3.80(3H, 2s); | |
| | 6.36-8.58(12H, m); 9.89-10.62 | |
| | (1H, 2s split, | |
| | broadened) | |
| 37b | (DMSO-d6)=2.70-2.89 | — |
| | (6H, s); 6.51-8.69(12H, | |
| | m); 9.58-10.58(1H, 2s | |
| | split, broadened) | |
| 38b | (DMSO-d6)=2.23-2.88 | — |
| | (3H, 2s); 6.90-8.27(12H, | |
| | m); 10.20-10.53(1H, 2s | |
| | broadened) | |
| 39b | (DMSO-d6)=2.21-2.79 | — |
| | (3H, 2s); 6.90-8.29(12H, | |
| | m); 10.18-1.57(1H, 2s | |
| | broadened) | |
| 40b | (DMSO-d6)=2.18-2.82 | — |
| | (3H, 2s); 6.95-8.31(12H, | |
| | m); 10.20-10.45(1H, 2s | |
| | broadened) | |
| 41b | (DMSO-d6)=3.68(3H, | — |
| | 2s); 4.49(2H, m); 6.76-8.32 | |
| | (13H, m); 9.09(1H, | |
| | very m broadened) | |
| 42b | (DSMO-d6)=4.49(2H, | — |
| | m); 6.79-8.35(13H, m); | |
| | 9.00-9.27(1H, 2m broadened) | |
| 43b | (DMSO-d6)=2.32-2.69 | — |
| | (3H, 2s); 4.48(2H, m); | |
| | 7.02-8.25(13H, m); 8.76-9.22 | |
| | (1H, 2m broadened) | |
| 44b | (DMSO-d6)=4.49(2H, | — |
| | m); 6.89-8.22(13H, m); | |
| | 8.96-9.33(1H, 2m broadened) | |
| 45b | (DMSO-d6)=4.49(2H, | — |
| | m); 7.01-8.31(13H, m); | |
| | 8.91-9.40(1H, 2m broadened) | |
| 46b | (DMSO-d6)=2.11-3.68 | — |
| | (6H, s); 4.42-4.52(2H, | |
| | 2m); 6.55-7.88(12H, m); | |
| | 8.54-8.88(1H, 2m broadened) | |
| 47b | (DMSO-d6)=6.71-8.26 | — |
| | (13H, m); 10.11-10.52 | |
| | (1H, 2s broadened) | |
| 48b | (DMSO-d6)=6.86-8.40 | — |
| | (13H, m); 10.08-10.63 | |
| | (1H, 2 s broadened) | |
| 49b | (DMSO-d6)=7.01-8.34 | — |
| | (12H, m); 10.49-11.03 | |
| | (1H, 2s broadened) | |
| 50b | (DMSO-d6)=3.85(3H, | — |
| | 2s); 6.79-8.32(12H, m); | |
| | 9.99-10.56(1H, 2s | |
| | broadened) | |
| 51b | (DMSO-d6)=6.93-8.36 | — |
| | (12H, m); 10.20-10.60 | |
| | (1H, 2s broadened) | |
| 52b | (DMSO-d6)=2.21-3.28 | — |
| | (9H, m), 6.78-8.64(12H, | |
| | m); 10.25-10.70(1H, 2s | |
| | broadened) | |
| 53b | (DMSO-d6)=4.51(2H, | — |
| | m); 6.99-8.33(12H, m); | |
| | 8.99-9.46(1H, 2m broadened) | |
| 54b | (DMSO-d6)=0.97-3.10 | — |
| | (12H, m); 3.82(3H, 2s); | |
| | 6.84-8.17(8H, m); 8.47-8.86 | |
| | (1H, 2m broadened) | |
| 55b | (DMSO-d6)=1.70-3.30 | — |
| | (4H, m); 3.71-4.70(3H, 2s+2H, | |
| | m); 6.71-8.21(11H, | |
| | m); 8.46-8.78(1H, 2m | |
| | broadened) | |
| 56b | (DSMO-d6)=1.31-2.96 | — |
| | (14H, m); 3.81(3H, 2s); | |
| | 6.93-8.27(8H, m); 8.47-8.87 | |
| | (1H, 2m broadened) | |
| 57b | (DMSO-d6)=0.88-1.31 | — |
| | (12H, m); 2.60-3.90(6H, | |
| | m); 3.81(3H, 2s); 6.86-8.13 | |
| | (8H, m); 8.59(1H, | |
| | 2m broadened) | |
| 58b | (DMSO-d6)=3.82(3H, | — |
| | 2s); 6.97-9.33(11H, m); | |
| | 10.71(1H, 2s broadened) | |
| 59b | (DMSO-d6)=2.11-3.90 | — |
| | (11H, m); 6.57-8.70(12H, | |
| | m); 9.94-10.30(1H, 2s | |
| | broadened) | |
| 60b | (DMSO-d6)=2.66-3.20 | — |
| | (4H, m); 3.53-3.92(4H, | |
| | m); 6.72-8.79(12H, m); | |
| | 10.04-10.73(1H, 2s | |
| | broadened) | |
| 61b | (DMSO-d6)=3.68(3H, | — |
| | 2s); 4.49(2H, m); 6.73-8.64 | |
| | (12H, m); 9.16(1H, | |
| | 2m broadened) | |
| 62b | (DMSO-d6)=2.09-2.91 | — |
| | (6H, m); 3.40(3H, s); | |
| | 6.84-8.87(12H, m) | |
| 63b | (DMSO-d6)=3.81(3H, | — |
| | 2s); 4.48(2H, m); 6.59-8.19 | |
| | (13H, m); 8.73-9.31 | |
| | (1H, 2m broadened) | |
| 64b | (DMSO-d6)=2.11-3.06 | — |
| | (6H, 2s); 4.48(2H, m); | |
| | 6.62-8.34(13H, m); 8.92-9.40 | |
| | (1H, 2m broadened) | |
| 65b | (DMSO-d6)=4.50(2H, | — |
| | m); 6.61-8.29(12H, m); | |
| | 8.95-9.39(1H, 2m broadened) | |

TABLE I

| Example No. | ¹H-NMR | LC-MS |
|---|---|---|
| 66a | — | (ES+) = 422.4(M + H) |
| 67a | — | (ES+) = 426.3(M + H) |
| 68a | — | (ES+) = 422.4(M + H) |
| 69a | — | (ES+) = 476.4(M + H) |
| 70a | — | (ES+) = 406.4(M + H) |
| 71a | — | (ES+) = 410.4(M + H) |
| 72a | — | (ES+) = 338.2(M + H) |
| | | 350.3(M + Na) |
| 73a | — | (ES+) = 342.2/344.2 with |
| | | a chlorine atom |
| | | 354.2/356.2 adduct of Na, |
| | | with a chlorine atom |
| 74a | — | (ES+) = 356.2/358.2 with |
| | | a chlorine atom |
| | | 368/370 with an adduct of |
| | | Na |
| | | (ES−) = 354.2/356.2 with a |
| | | chlorine atom |
| 75a | — | (ES+) = 352.1(M + H) |
| | | 364.1(M + Na) |
| 76a | — | (ES+) = 390.1(M + H) |
| | | (ES−) = 388.1(M − H) |
| 77a | — | (ES+) = 336.2(M + H) |
| 78a | — | (ES+) = 391.2(M + H) |
| 79a | — | (ES+) = 375.2(M + H) |
| | | (ES−) = 373.2(M − H) |
| 80a | — | (ES+) = 321.3(M + H) |
| 81a | — | (ES+) = 380.3(M + H) |
| 82a | — | (ES+) = 384.2/386.2 |
| | | (M + H) with a chlorine |
| | | atom |
| 83a | — | (ES+) = 396.3(M + H) |
| 84a | — | (ES+) = 434.3(M + H) |
| | | (ES−) = 432.3(M − H) |
| 85a | — | (ES+) = 364.3(M + H) |
| 86a | — | (ES+) = 368.3(M + H) |
| 87a | — | (ES+) = 371.3(M + H) |
| | | (ES−) = 369.3(M − H) |
| 88a | — | (ES+) = 419.3(M + H) |
| 89a | — | (ES+) = 423.3(M + H) |
| | | (ES−) = 421.3(M − H) |
| 90a | — | (ES+) = 460.4(M + H) |
| 91a | — | (ES+) = 392.2(M + H) |
| | | (ES−) = 390.2(M − H) |
| 92a | — | (ES−) = 398.3(M − H) |
| 93a | — | (ES−) = 407.3(M − H) |
| 94a | — | (ES+) = 368.1(M + H) |
| | | (ES−) = 366.1(M − H) |
| 95a | — | (ES+) = 406.1(M + H) |
| | | (ES−) = 404.1(M − H) |
| 96a | — | (ES+) = 340.2(M + H) |
| | | (ES−) = 338.2(M − H) |
| 97a | — | (ES+) = 421.3(M + H) |
| | | (ES−) = 419.3(M − H) |
| 98a | — | (ES−) = 471.3(M − H) |

TABLE J

| Example No. | ¹H-NMR | LC-MS |
|---|---|---|
| 66b | (DMSO-d6)=0.87-3.99 | — |
| | (8H, m+3H, 2s); 6.48-8.19 | |
| | (11H, m); 10.01-10.82 | |
| | (1H, 2s split, | |
| | broadened) | |
| 67b | (DMSO-d6)=2.0-3.14 | — |
| | (4H, m); 3.61-3.87(4H, | |
| | m); 6.51-8.09(11H, m); | |
| | 9.69-10.43(1H, 2s | |
| | broadened) | |
| 68b | (DMSO-d6)=1.19-4.09 | — |
| | (11H, m); 6.42-8.54(11H, | |
| | m) | |
| 69b | (DMSO-d6)=0.90-3.89 | — |
| | (8H, m); 6.67-8.03(11H, | |
| | m); 10.27(1H, 2s broadened) | |
| 70b | (DMSO-d6)=0.87-3.84 | — |
| | (8H, m+3H, 2s); 6.55-8.00 | |
| | (11H, m); 10.08-10.39 | |
| | (1H, 2s broadened) | |
| 71b | (DMSO-d6)=0.91-3.85 | — |
| | (8H, m); 6.58-7.96(11H, | |
| | m); 9.94-10.40(1H, 2s | |
| | split, broadened) | |
| 72b | (DMSO-d6)=3.70(3H, | — |
| | 2s); 6.83-8.62(11H, m); | |
| | 10.13-10.88(1H, 2s | |
| | broadened) | |
| 73b | (DMSO-d6)=6.50-8.51 | — |
| | (11H, m); 10.15-10.91 | |
| | (1H, 2s split, broadened) | |
| 74b | (DMSO-d6)=4.51(2H, | — |
| | m); 6.68-8.65(11H, m) | |
| 75b | (DMSO-d6)=3.90(3H, | — |
| | 2s); 4.49(2H, m); 6.60-8.80 | |
| | (11H, m) | |
| 76b | (DMSO-d6)=4.50(2H, | — |
| | m); 6.82-8.75(11H, m); | |
| | 8.85-9.30(1H, 2s split, | |
| | broadened) | |
| 77b | (DMSO-d6)=2.36(3H, | — |
| | 2s); 4.49(2H, m); 6.86-8.63 | |
| | (11H, m); 9.11(1H, | |
| | 2s broadened) | |
| 78b | (DMSO-d6)=6.70-8.02 | — |
| | (12H, m); 10.35-10.66 | |
| | (1H, 2s broadened) | |
| 79b | (DMSO-d6)=6.89-8.27 | — |
| | (12H, m); 10.37-10.69 | |
| | (1H, 2s broadened) | |
| 80b | (DMSO-d6)=2.33(3H, | — |
| | 2s); 6.75-8.02(12H, m); | |
| | 9.97-10.64(1H, 2s | |
| | broadened) | |
| 81b | (DMSO-d6)=2.0-3.99 | — |
| | (9H, m); 6.46-8.14(11H, | |
| | m); 9.87-10.81(1H, 2s | |
| | split, broadened) | |
| 82b | (DMSO-d6)=2.27-3.92 | — |
| | (6H, m); 6.64-8.08(11H, | |
| | m); 9.95-10.44(1H, 2s | |
| | split, broadened) | |
| 83b | (DMSO-d6)=2.78(3H, | — |
| | 2s); 2.85-3.93(6H, m); | |
| | 6.56-8.11(11H, m); 9.95-10.87 | |
| | (1H, 2s split, | |
| | broadened) | |
| 84b | (DMSO-d6)=2.56-3.60 | — |
| | (6H, m); 7.04-8.01(11H, | |
| | m); 9.93-10.76(1H, 2s | |
| | split, broadened) | |
| 85b | (DMSO-d6)=2.19-3.78 | — |
| | (9H, m); 6.40-8.04(11H, | |
| | m); 9.88-10.79(1H, 2s | |
| | split, broadened) | |
| 86b | (DMSO-d6)=2.72-3.92 | — |
| | (6H, m); 6.98-8.02(11H, | |
| | m); 10.29-10.89(1H, 2s | |
| | split, broadened) | |
| 87b | (DMSO-d6)=2.24-3.78 | — |
| | (3H, 2s); 7.04-8.16(11H, | |
| | m); 9.89-10.75(1H, 2s | |
| | split, broadened) | |
| 88b | (DMSO-d6)=2.25-3.62 | — |
| | (3H, 2s); 4.48(2H, m); | |
| | 6.66-8.15(11H, m); 8.64-9.33 | |
| | (1H, m) | |
| 89b | (DMSO-d6)=4.46(2H, | — |
| | m); 6.26-8.05(11H, m); | |
| | 8.12-8.75(1H, m | |
| | broadened) | |
| 90b | (DMSO-d6)=1.10-4.30 | — |
| | (8H, m); 6.83-8.04(11H, | |
| | m); 9.12-10.24(1H, 2m | |
| | broadened) | |
| 91b | (DMSO-d6)=7.06-8.62 | — |
| | (11H, m); 10.49-10.89 | |
| | (1H, 2s split, broadened) | |
| 92b | (DMSO-d6)=6.73-8.40 | — |
| | (11H, m); 10.78-11.22 | |
| | (1H, 2s broadened) | |
| 93b | (DMSO-d6)=6.71-8.27 | — |
| | (11H, m); 9.82-10.81(1H, | |
| | 2s split, broadened) | |
| 94b | (DMSO-d6)=2.52(3H, | — |
| | 2s); 4.47(2H, m); 6.82-8.61 | |
| | (11H, m); 8.88-9.32 | |
| | (1H, m broadened) | |
| 95b | (DMSO-d6)=4.48(2H, | — |
| | m); 7.00-8.61(11H, m); | |
| | 8.70-9.23(1H, split, m | |
| | broadened) | |
| 96b | (DMSO-d6)=4.48(2H, | — |
| | m); 6.81-8.58(11H, m) | |
| 97b | (DMSO-d6)=1.44-3.95 | — |
| | (3H, 2s); 6.76-8.22(11H, | |
| | m); 10.33-10.73(1H, 2s | |
| | broadened) | |
| 98b | (DMSO-d6)=4.48(2H, | — |
| | m); 6.66-8.26(11H, m); | |
| | 8.80-9.28(1H, 2m broadened) | |

TABLE K

| Example No. | ¹H-NMR | LC-MS |
|---|---|---|
| 99a | — | (ES+) = 418.4(M + H) |
| 100a | — | (ES+) = 422.2(M + H) |
| 101a | — | (ES+) = 418.4(M + H) |
| 102a | — | (ES+) = 434.3(M + H) |
| 103a | — | (ES+) = 402.4(M + H) |
| 104a | — | (ES+) = 406.4(M + H) |
| 105a | — | (ES+) = 351.3(M + H) |
| | | (ES−) = 349.3(M − H) |
| | | 395.3(M + HCOO-adduct) |
| 106a | — | (ES+) = 339.3(M + H) |
| | | (ES−) = 337.3(M − H) |
| | | 383.3(M + HCOO-adduct) |
| 107a | — | (ES+) = 430.3(M + H) |
| 108a | — | (ES+) = 364.3(M + H) |
| 109a | — | (ES+) = 379.3(M + H) |
| 110a | — | (ES+) = 383.3(M + H) |
| 111a | — | (ES+) = 363.3(M + H) |
| 112a | — | (ES+) = 351.3(M + H) |
| 113a | — | (ES+) = 347.4(M + H) |
| 114a | — | (ES+) = 363.3(M + H) |
| 115a | — | (ES+) = 331.4(M + H) |
| 116a | — | (ES+) = 431.4(M + H) |
| 117a | — | (ES+) = 435.3/437.3 |
| | | (M + H with a chlorine |
| | | atom) |
| | | (ES−) = 433.3/435.3(M − H |
| | | with a chlorine atom) |
| 118a | — | (ES+) = 447.3(M + H) |
| 119a | — | (ES+) = 485.3(M + H) |
| 120a | — | (ES+) = 419.3(M + H) |
| | | (ES−) = 463.4(M + HCOO- |
| | | adduct) |
| 121a | — | (ES+) = 321.3(M + H) |
| | | (ES−) = 319.3(M − H) |
| | | 365.3(M + HCOO-adduct) |
| 122a | — | (ES+) = 351.3(M + H) |
| | | (ES−) = 349.3(M − H) |
| | | 395.3(M + HCOO-adduct) |
| 123a | — | (ES+) = 355.3/357.3 |
| | | (M + H with a chlorine |
| | | atom) |
| | | (ES−) = 353.3/355.3(M − H |
| | | with a chlorine atom) |
| | | 399.3/401.3(M − HCOO- |
| | | adduct with a chlorine |
| | | atom) |
| 124a | — | (ES+) = 405.3(M + H) |
| | | (ES−) = 403.3(M − H) |
| | | 449.3(M + HCOO-adduct) |
| 125a | — | (ES+) = 335.3(M + H) |
| | | (ES−) = 333.3(M − H) |
| | | 379.3(M + HCOO-adduct) |
| 126a | — | (ES+) = 392.3(M + H) |
| 127a | — | (ES+) = 395.3(M + H) |
| 128a | — | (ES+) = 347.3(M + H) |
| 129a | — | (ES+) = 401.3(M + H) |
| 130a | — | (ES+) = 415.4(M + H) |

TABLE L

| Example No. | ¹H-NMR | LC-MS |
|---|---|---|
| 99b | (DMSO-d6)=0.5-4.72(13H, | — |
| | m); 6.71-8.20(12H, m); | |
| | 10.48(1H, 2s broadened) | |
| 100b | (DMSO-d6)=0.7-4.0(10H, | — |
| | m); 6.73-7.88(12H, m); | |
| | 9.86-10.65(1H, split, s | |
| | broadened) | |
| 101b | (DMSO-d6)=0.71-3.76 | — |
| | (10H, m); 3.80(3H, 2s); | |
| | 6.69-7.88(12H, m); 9.80-10.68 | |
| | (1H, 2s split, | |
| | broadened) | |
| 102b | (DMSO-d6)=0.71-3.86 | — |
| | (10H, m); 3.01(3H, s); | |
| | 6.71-7.97(12H, m); 9.84-10.13 | |
| | (1H, 2s split, | |
| | broadened) | |
| 103b | (DMSO-d6)=0.72-3.81 | — |
| | (10H. m); 2.34(3H, 2s); | |
| | 6.67-7.88(12H, m); 9.84-10.80 | |
| | (1H, 2s split, | |
| | broadened) | |
| 104b | (DMSO-d6)=0.70-3.95 | — |
| | (10H, m); 6.70-7.87(12H, | |
| | m); 9.82-10.21(1H, 2s | |
| | split, broadened) | |
| 105b | (DMSO-d6)=3.73-4.02(2H, | — |
| | 2s+3H, 2s); 6.90-7.94 | |
| | (12H, m); 10.12-10.40(1H, | |
| | 2s split, broadened) | |
| 106b | (DMSO-d6)=3.37-3.85 | — |
| | (2H, 2s); 6.85-8.20(12H, | |
| | m); 10.06-10.50(1H, 2s | |
| | split, broadened) | |
| 107b | (DMSO-d6)=2.76-3.84 | — |
| | (8H, m); 6.50-7.96(12H, | |
| | m); 9.83-10.57(1H, 2s | |
| | split, broadened) | |
| 108b | (DMSO-d6)=3.05-4.00 | — |
| | (8H, m); 6.84-7.90(12H, | |
| | m); 10.05-10.58(1H, 2s | |
| | split, broadened) | |
| 109b | (DMSO-d6)=2.0-4.38(8H, | — |
| | m); 7.06-8.16(12H, m); | |
| | 10.02-10.65(1H, 2s split, | |
| | broadened) | |
| 110b | (DMSO-d6)=2.0-4.38(5H, | — |
| | m); 7.04-8.04(12H, m); | |
| | 9.99-10.70(1H, 2s split, | |
| | broadened) | |
| 111b | (DMSO-d6)=2.0-4.27(8H, | — |
| | m); 6.94-7.93(12H, m); | |
| | 10.03-10.65(1H, 2s split, | |
| | broadened) | |
| 112b | (DMSO-d6)=3.57(2H, 2s); | — |
| | 3.98-4.46(2H, 2s); 6.69-7.93 | |
| | (13H, m) | |
| 113b | (DMSO-d6)=3.48-3.86 | — |
| | (3H, 2s, +2H, 2s); 4.27 | |
| | (2H, 2s); 6.98-7.97(13H, | |
| | m); 8.42-9.26(1H, 2s split, | |
| | broadened) | |
| 114b | (DMSO-d6)=2.0-3.63(3H, | — |
| | 2s+2H, 2s); 4.27(2H, 2s); | |
| | 6.93-8.29(13H, m); 8.40-9.46 | |
| | (1H, 2s split, | |
| | broadened) | |
| 115b | (DMSO-d6)=2.0-3.76(2H, | — |
| | 2s+3H, 2s); 4.26(2H, 2s); | |
| | 6.82-7.97(13H, m) | |
| 116b | (DMSO-d6)=3.41-4.19(3H, | — |
| | 2s+2H, 2s); 4.28(2H, 2s); | |
| | 6.69-7.92(12H, m) | |
| 117b | (DMSO-d6)=3.64-4.40(2H, | — |
| | 2s); 4.29(2H, 2s); 6.88-8.00 | |
| | (12H, m); 8.68(1H, 2s | |
| | split, broadened) | |
| 118b | (DMSO-d6)=2.02-4.18(2H, | — |
| | 2s+3H, 2s); 4.29(2H, m); | |
| | 6.80-8.19(12H, m); 8.63 | |
| | (1H, 2s split, broadened) | |
| 119b | (DMSO-d6)=3.57(2H, 2s); | — |
| | 4.29(2H, m); 6.92-7.96 | |
| | (12H, m); 8.68(1H, 2s split, | |
| | broadened) | |
| 120b | (DMSO-d6)=3.55(2H, 2s); | — |
| | 4.29(2H, m); 6.83-7.91 | |
| | (12H, m); 8.63(1H, m) | |
| 121b | (DMSO-d6)=3.52-3.82 | — |
| | (2H, 2s); 6.87-7.74(13H, | |
| | m); 10.15(1H, 2s split, | |
| | broadened) | |
| 122b | (DMSO-d6)=3.56-4.20 | — |
| | (2H, 2s+3H, 2s); 6.92-7.83 | |
| | (12H, m); 10.21(1H, | |
| | 2s split, broadened) | |
| 123b | (DMSO-d6)=3.56-4.21 | — |
| | (2H, 2s); 6.93-7.76(12H, | |
| | m); 10.23(1H, 2s split, | |
| | broadened) | |
| 124b | (DMSO-d6)=3.57-4.27 | — |
| | (2H, 2s); 6.84-8.20(12H, | |
| | m); 10.23(1H, 2s split, | |
| | broadened) | |
| 125b | (DMSO-d6)=3.56-4.23 | — |
| | (2H, 2s); 6.91-7.90(12H, | |
| | m); 10.22(1H, 2s broadened) | |
| 126b | (DMSO-d6)=2.00-3.92 | — |
| | (11H, m); 6.86-7.92(12H, | |
| | m); 10.39(1H, 2s split, | |
| | broadened) | |
| 127b | (DMSO-d6)=2.0-4.30(8H, | — |
| | m); 6.87-7.91(12H, m); | |
| | 10.07-10.56(1H, 2s split, | |
| | broadened) | |
| 128b | (DMSO-d6)=3.46-3.85 | — |
| | (2H, 2s+3H, 2s); 4.26 | |
| | (2H, m); 6.78-7.93(13 H, | |
| | m); 8.58(1H, m | |
| | broadened) | |
| 129b | (DMSO-d6)=3.43-3.60 | — |
| | (2H, 2s); 4.27(2H, m); | |
| | 6.96-7.84(13H, m); 8.59 | |
| | (1H, m broadened) | |
| 130b | (DMSO-d6)=2.11-2.42 | — |
| | (3H, 2s); 3.40-3.63(2H, 2s | |
| | split); 4.28(2H, m); 6.76-8.27 | |
| | (12H, m); 8.59(1H, m | |
| | broadened) | |

TABLE M

| Example No. | ¹H-NMR | LC-MS |
|---|---|---|
| 131a | — | (ES+)=337.25(M+H) |
| | | (ES−)=335.27(M−H) |
| 132a | (DMSO-d6)=3.71(3H, s); | — |
| | 4.44(2H, m); 6.55-8.22(11H, | |
| | m); 8.22-8.69(2H, m); | |
| | 8.95(1H, m) | |
| 133a | (DMSO-d6)=3.72(3H, s); | — |
| | 6.73-7.74(8H, m); 8.12(1H, | |
| | s); 8.91(1H, s); 9.14(2H, | |
| | s); 10.53(1H, s | |
| | broadened) | |
| 134a | (DMSO-d6)=3.72(3H, s); | — |
| | 6.73-7.67(9H, m); 7.67-7.96 | |
| | (1H, m); 7.97-8.26 | |
| | (2H, m); 8.36(1H, s); 10.59 | |
| | (1H, s broadened) | |
| 135a | (DMSO-d6)=3.72(3H, s); | — |
| | 6.69-7.51(8H, m); 7.51-7.90(2H, | |
| | m); 8.11(1H, m); 8.44(2H, m); | |
| | 10.49(1H, s broadened) | |

TABLE N

| Example No. | ¹H-NMR | LC-MS |
|---|---|---|
| 131b | (DMSO-d6)=4.45(2H, m); | — |
| | 7.2-8.15(13H, m); 9.25(1H, | |
| | 2t, J = 5.7 Hz) | |
| 132b | (DMSO-d6)=3.81(3H, 2s); | — |
| | 4.49(2H, m); 6.96-8.08(10H, | |
| | m); 8.27-8.73(2H, m); | |
| | 9.27(1H, m) | |
| 133b | (DMSO-d6)=3.82(3H, 2s); | — |
| | 6.66-8.36(8H, m); 8.94(1H, | |
| | s); 9.15(2H, m); 10.79(1H, | |
| | s broadened) | |
| 134b | (DMSO-d6)=3.82(3H, 2s); | — |
| | 6.66-8.62(12H, m); 10.96(1H, | |
| | 2s broadened) | |
| 135b | (DMSO-d6)=3.82(3H, 2s); | — |
| | 6.93-8.28(10H, m); 8.48(2H, | |
| | m); 10.73(1H, s broadened) | |

TABLE O

| Example No. | ¹H-NMR | LC-MS |
|---|---|---|
| 136a | (DMSO-d6)=3.72(3H, s); | — |
| | 6.67-7.38(7H, m); 7.41-7.58(3H, | |
| | m); 8.06-8.41(2H, m); | |
| | 8.55(1H, s); 10.24 | |
| | (1H, s broadened) | |
| 137a | (DMSO-d6)=6.97-8.03 | — |
| | (10H, m); 8.04-8.49(2H, | |
| | m); 9.36(1H, s); 10.42(1H, s | |
| | broadened) | |
| 138a | (DMSO-d6)=3.74(3H, s); | — |
| | 3.96(2H, m); 6.77-7.96(11H, | |
| | m); 8.27-8.60(3H, | |
| | m) | |

TABLE P

| Example No. | ¹H-NMR | LC-MS |
|---|---|---|
| 136b | (DMSO-d6)=3.80(3H, s); | — |
| | 6.88-8.06(10H, m); 8.06-8.54(2H, | |
| | m); 10.66(1H, s broadened) | |
| 137b | (DMSO-d6)=7.04-8.55(12H, | — |
| | m); 10.71(1H, s broadened) | |
| 138b | (DMSO-d6)=3.82(3H, s); | — |
| | 3.96-4.18(2H, m); 7.01-7.97(10H, | |
| | m); 8.23-8.54(3H, m) | |

TABLE Q

| Example No. | ¹H-NMR | LC-MS |
|---|---|---|
| 139a | (DMSO-d6)=3.75(3H, s); | — |
| | 6.79-7.09(6H, m); 7.09-7.21(1H, | |
| | m); 7.22-7.41(2H, | |
| | m); 7.42-7.59(1H, m); | |
| | 8.27(3H, m); 10.44(1H, s | |
| | broadened) | |

TABLE R

| Example No. | ¹H-NMR | LC-MS |
|---|---|---|
| 139b | (DMSO-d6)=3.82(3H, m); | — |
| | 6.65-8.03(10H, m); 8.26(2H, | |
| | m); 10.64(1H, s broadened) | |

TABLE S

| Examples | Structures | NMR | LC-MS |
|---|---|---|---|
| 140a | 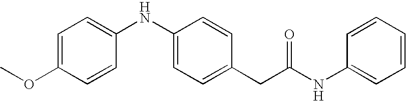 | | M+H = 333 |
| 141a | 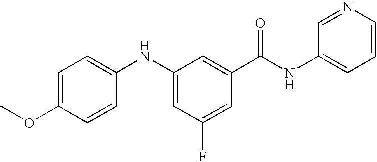 | (DMSO-d6): 3.73 (3H, m); 6.65-7.71 (8H, m); 8.03-8.47 (3H, m); 8.74-9.03 (1H, m); 10.38 (1H, s broad). | |
| 140b | 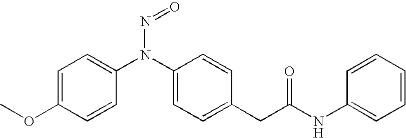 | (DMSO-d6): 3.68 and 3.71 (2H, 2s); 3.78 and 3.80 (3H, 2s); 6.91-7.80 (13H, m); 10.04-10.36 (1H, m). | |
| 141b | 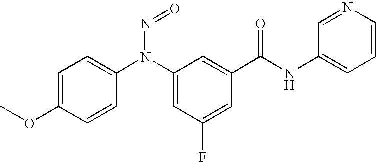 | (DMSO-d6): 3.80 and 3.82 (3H, 2s); 6.97-8.44 (10H, m); 8.77-9.03 (1H, m); 10.61 (1H, s broad). | |

1. Compound of the formula I: 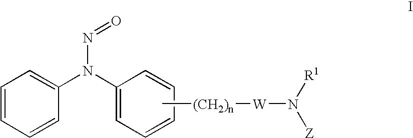
in which
each of the phenyl rings represented is optionally substituted one or more times;
n represents an integer selected from 0, 1, 2, 3, 4 and 5;
W represents —CO— or —SO₂—;
Z represents H; alkyl; aryl; or arylalkyl;
R₁ represents any monovalent organic group;
and the pharmaceutically acceptable salts thereof.

2. Compound according to claim 1 of the formula I, in which:
R¹ represents -A-Cy in which A represents a bond, alkylene or alkenylene; and Cy represents aryl, which is optionally substituted by one or more radicals St; heteroaryl, which is optionally substituted by one or more radicals St; or a saturated and/or unsaturated heterocycle, which is optionally substituted by one or more radicals St; or alternatively R¹ represents -A-NRₐR_{b}, in which A is as defined above; Rₐ represents H or alkyl; and R_{b} represents alkyl;
St is selected from nitro; a halogen atom; cyano; optionally halogenated alkylthio; alkylamino; dialkylamino; optionally halogenated alkyl; optionally halogenated alkoxy; a saturated and/or unsaturated heterocycle, which is optionally substituted by alkyl or alkoxy.

3. Compound of the formula Ia: 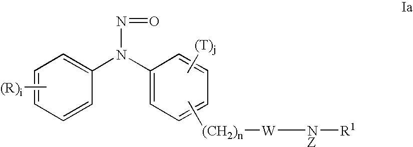
in which
W represents —CO— or SO₂—;
n represents an integer selected from 0, 1, 2, 3, 4 and 5;
i represents an integer selected-from 0, 1, 2, 3, 4 and 5;
R, which may be identical or different, represent optionally halogenated alkoxy; optionally halogenated alkylthio; optionally halogenated alkyl; optionally halogenated alkylsulfonyl; halogen; dialkylamino; cyano; alkylamino; or nitro;
Z represents H; alkyl; aryl; or arylalkyl;
T represents H or a halogen atom; or an alkyl group; an alkoxy group; an alkylthio group; an alkylamino group; or a dialkylamino group;
j represents an integer selected from 0, 1, 2, 3 and 4;
R¹ is as defined in claim 1; and the pharmaceutically acceptable salts thereof.

4. Compound according to claim 1, characterised in that R¹ represents optionally substituted phenyl; —(CH₂)ᵣ-Ph^{o}, in which Ph^{o} is optionally substituted and r represents an integer selected from 1, 2 and 3, preferably 1; —B-phenyl, in which B represents C₂-C₅ alkenylene; —(CH₂)ₜ-Het, in which t is an integer selected from 0, 1, 2 and 3; and Het represents an optionally substituted saturated and/or unsaturated aromatic heterocycle, preferably monocyclic, containing 1 to 3 hetero atoms selected from N, O and S, or Het represents quinuclidine; —(CH₂)ₛ—NRₐR_{b}, in which s is an integer selected from 0, 1 and 2 and Rₐ and R_{b}, which may be identical or different, are alkyl.

5. Compound according to claim 4, characterised in that R¹ represents —(CH₂)ₜ-Het in which Het is a radical selected from pyridyl; imidazolyl; piperidyl; piperazinyl; and pyrimidyl, the said heterocycle being optionally substituted.

6. Compound according to claim 1, characterised in that Z represents H.

7. Compound according to claim 1, characterised in that W represents SO₂; R¹ represents —(CH₂)ₜ-Het, in which t represents an integer selected from 0, 1, 2, 3 and 4 and Het represents an aromatic heterocycle, which is preferably monocyclic, containing 1 to 3 hetero atoms selected from O, N and S, the said heterocycle optionally being substituted.

8. Compound according to claim 7, characterised in that Het represents pyridyl and t is 0 or 1.

9. Compound according to claim 1, characterised in that W is —CO—; and R¹ represents —(CH₂)ₜ-Het in which t is an integer selected from 0, 1, 2 and 3; and Het represents an aromatic heterocycle, which is preferably monocyclic, containing 1 to 3 hetero atoms selected from O, N and S, the said heterocycle optionally being substituted.

10. Compound according to claim 9, characterised in that Het is pyridyl and t is 0 or 1.

11. Compound according to claim 1, characterised in that the group —(CH₂)ₙ—W—N(Z)—R¹ is in a meta position or in the para position relative to the —N—N═O group.

12. Process for preparing compounds of the formula I, which comprises the reaction of a compound of the formula II: 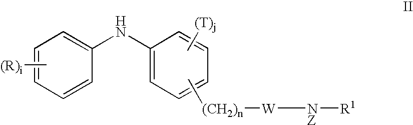
in which R, T, i, j, n, W, Z and R¹ are as defined in claim 3, with a nitrosating, agent, such as an alkali metal nitrite, in acidic medium.

13. Compound of the formula III: 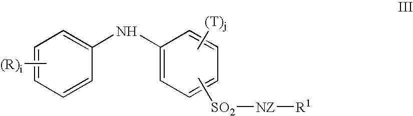
in which:
i, j, R, Z and T are as defined in claim 1;
R¹ represents phenyl, which is optionally substituted by one or more radicals St; —(CH₂)ᵣ-Ph^{o}, in which Ph^{o} is optionally substituted by one or more radicals St and r represents an integer selected from 1, 2 and 3, or alternatively R¹ represents —(CH₂)ₜ-Het, in which Het is a radical selected from pyridyl; imidazolyl; piperidyl; piperazinyl; and pyrimidyl, the said radical optionally being substituted by one or more radicals St and t is selected from an integer 0, 1, 2 and 3; with the exclusion of the following compounds defined by formula III in which:
a) R in position 2=R in position 4=NO₂; i=2; j=0; Z=H; and R¹=2-pyridyl; or
b) R in position 2=R in position 4=NO₂; i=2; j=0; Z=H; and R¹ represents 2,6-dimethyl-4-pyrimidyl, or 4,6-dimethyl-2-pyrimidyl;
c) R¹ represents phenyl; Z=H; i=0,1; j=0; and R represents diethylamino;
d) R¹ represents 2,4-dinitrophenyl; i=2; R in position 2=R in position 4=NO₂; j=0; Z=H;
e) R¹ represents 2,4,6-triisopropylphenyl; Z=H; i=1; j=0; R=di(n-hexyl)amino;
f) R in position 2=-R in position 6=R in position 4=NO₂; i=3; j=0; Z=H; R¹=2,6-dimethoxy-4-pyrimidyl.

14. Compound of the formula III 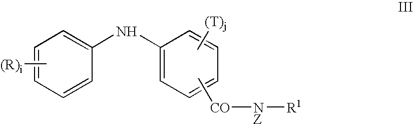
in which:
i, j, R, Z and T areas defined in claim 1;
R¹ represents phenyl, which is optionally substituted by one or more radicals St; —(CH₂)ᵣ-Ph^{o}, in which Ph^{o} is optionally substituted by one or more radicals St and r represents an integer selected from 1, 2 and 3; or R¹ represents —(CH₂)ₜ-Het, in which Het is a radical selected from pyridyl; imidazolyl; piperidyl; piperazinyl; and pyrimidyl, the said radical optionally being substituted by one or more radicals St, St being as defined in claim 2, and t is selected from an integer 0, 1, 2 and 3; with the exclusion of the following compounds defined by formula III in which:
a) R¹=4-methyl-3-nitrophenyl; 4-ethoxyphenyl; 2-bromo-4-nitrophenyl; phenyl; 4-bromophenyl; 2-chlorophenyl; 3-fluorophenyl; 4-methoxyphenyl; 2-methoxyphenyl; 4-dimethylaminophenyl; 3-methoxyphenyl; 2,4-dinitrophenyl; 4-methylphenyl; 3-methylphenyl; or 2-methylphenyl; i=2,3; R=NO₂; j=0;
b) R¹=2-pyridyl; i=3; R=NO2; j=0.

15. Compound of the formula IV: 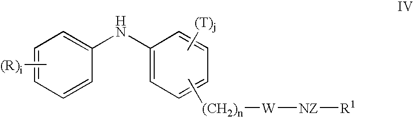
in which:
W represents —CO— or —SO₂—;
R, Z, T, I and j are as defined in claim 3;
R¹ represents phenyl, which-is optionally substituted by one or more radicals St;
—(CH₂)ᵣ-Ph^{o}, in which Ph^{o} is optionally substituted by one or more radicals St, St being as defined in claim 2, and r represents an integer selected from 1, 2 and 3; or R¹ represents —(CH₂)ₜ-Het, in which Het is a radical selected from pyridyl; imidazolyl; piperidyl; piperazinyl; and pyrimidyl, the said radical optionally being substituted by one or more radicals St and t is selected from the integers 0, 1, 2 and 3.

16. Pharmaceutical composition comprising at least one compound of the formula I according to claim 1 in combination with one or more pharmaceutically acceptable excipients.

17. Pharmaceutical composition comprising at least one compound of the formula III or IV according to claim 13, respectively, in combination with one or more pharmaceutically acceptable excipients.

18. Use of a compound of the formula I according to claim 1, for the preparation of a medicament that can be used in the treatment of pathologies that are characterised by an oxidative stress condition and a lack of availability of endothelial nitrogen monoxide.

19. Use of a compound of the formula III or IV according claim 13, respectively, in combination with one or more pharmaceutically acceptable excipients for the preparation of an antioxidant medicament that can be used as a free-radical scavenger.

20. Use of a compound of the formula I according to claim 1, or of a compound of the formula II as defined in claim 12, for the preparation of a medicament that can be used in the treatment of metabolic insulin resistance syndrome.
